SEQUENCE LISTING (38 sequences)

SEQ ID NO: 1 - DNA, 30 bp, M13 bacteriophage (modified)
  M13 bacteriophage (modified) 1-13
tatgaggctcttgaggatattgctactaac

SEQ ID NO: 2 - protein, 10 aa, M13 bacteriophage (modified)
  M13 bacteriophage (modified) 1-10
YEALEDIATN

SEQ ID NO: 3 - protein, 14 aa, Artificial sequence
  Peptide epitope flag
MADPNRFRGKDLGG

SEQ ID NO: 4 - DNA, 57 bp, Artificial sequence
  Mutagenic oligonucleotide
atccccaaggaacagrmakmttcattcsytcagaacscacagacctccctctgtttc

SEQ ID NO: 5 - DNA, 60 bp, Artificial sequence
  Mutagenic oligonucleotide
tcagaatcgattccgacascakccrmcsstgaggaarctsmacagaaatccaacctagag

SEQ ID NO: 6 - DNA, 78 bp, Artificial sequence
  Mutagenic oligonucleotide
aactacgggctgctckmytgcttcsstrmagacatggmtrmagtcgagrctkytctgsstrytgtgcagtgccgctct

SEQ ID NO: 7 - DNA, 57 bp, Artificial sequence
  Mutagenic oligonucleotide
atccccaaggaacagarmtmctcattctygcagaacyctcagacctccctctgtttc

SEQ ID NO: 8 - DNA, 57 bp, Artificial sequence
  Mutagenic oligonucleotide
gaatcgattccgacaycttccarcmgtgaggaawcgymgcagaaatccaacctagag

SEQ ID NO: 9 - DNA, 78 bp, Artificial sequence
  Mutagenic oligonucleotide
aactacgggctgctctmctgcttcmgtarmgacatgkmcarmgtckmgwcgtycctgmgtakcgtgcagtgccgctct

SEQ ID NO: 10 - DNA, 96 bp, Artificial sequence
  Mutagenic oligonucleotide
ataccactctcgaggctckctgacaacgcgtkgctgcgtgctgamcgtcttracsaactggcctwcgamacgtacsaagagtttgaagaagcctat

SEQ ID NO: 11 - DNA, 56 bp, Artificial sequence
  Mutagenic oligonucleotide
atcccaaaggaacagrttmactcattctkgtkgaacycgcagacctccctctgtcc

SEQ ID NO: 12 - DNA, 60 bp, Artificial sequence
  Mutagenic Oligonucleotide
tcagagtctattccgacaycgkccracarggamgaaacasaacagaaatccaacctagag

SEQ ID NO: 13 - DNA, 93 bp, Artificial sequence
  Mutagenic oligonucleotide
aagaactacgggttactctwctgcttcracarggacatgkccarggtckccasctwcctgargascgtgcagtgcargtctgtggagggcagc

SEQ ID NO: 14 - DNA, 93 bp, Artificial sequence
  Mutagenic oligonucleotide
tccgggagctccagcgstgmagstgmtgmtscagstrmagstgstkytrmckccsytsmagstkccgstrctgaatatatcggttatgcgtgg

SEQ ID NO: 15 - DNA, 87 bp, Artificial sequence
  Mutagenic oligonucleotide
ctgcaagcctcagcgaccgmakmtrytgstkmtgstksggstryggytgytgytrytgytgstgstrctatcggtatcaagctgttt

SEQ ID NO: 16 - DNA, 75 bp, Artificial sequence
  Mutagenic oligonucleotide
attgtcggcgcaactrytgstrytrmasytkytrmarmakytrctkccrmagstkcctgataaaccgatacaatt

SEQ ID NO: 17 - protein, 12 aa, Artificial sequence
  Peptide epitope flag.
MADPNRFRGKDL

SEQ ID NO: 18 - DNA, 48 bp, Artificial sequence
  Mutagenic oligonucleotide
acctgcaaggccagtsmagmtgtgkccrytgstgtcgcctggtatcaa

SEQ ID NO: 19 - DNA, 48 bp, Artificial sequence
  Mutagenic oligonucleotide
aaactactgatttackccgctkcckmtcgakmtactggagtcccttct

SEQ ID NO: 20 - DNA, 48 bp, Artificial sequence
  Mutagenic oligonucleotide
tattactgtcaacaakmtkmtrytkmtcctkmtacgtttggacagggt

SEQ ID NO: 21 - DNA, 66 bp, Artificial sequence
  Mutagenic oligonucleotide
gtcaccatcacctgcrmagstkcccaggatgyttctattggtgytgsttggtatcaacagaaacca

SEQ ID NO: 22 - DNA, 51 bp, Artificial sequence
  Mutagenic oligonucleotide
aaactactgatttactcggsttcctacssttacrctggagtcccttctcgc

SEQ ID NO: 23 - DNA, 57 bp, Artificial sequence
  Mutagenic oligonucleotide
gcaacttattactgtsmasmatattatatttatscatacrcttttggacagggtacc

SEQ ID NO: 24 - DNA, 48 bp, Artificial sequence
  Mutagenic oligonucleotide
gcagcttctggcttcrctttcrctgmtkmtrctatggactgggtccgt

SEQ ID NO: 25 - DNA, 69 bp, Artificial sequence
  Mutagenic oligonucleotide
ctggaatgggttgcagmtgytrmccctrmckccggcggctctryttatrmcsmacgcttcaagggccgt

SEQ ID NO: 26 - DNA, 45 bp, Artificial sequence
  Mutagenic oligonucleotide
tattattgtgctcgtrmcsytggascakccttctactttgactac

SEQ ID NO: 27 - DNA, 54 bp, Artificial sequence
  Mutagenic oligonucleotide
gcagcttctggcttcaccttcaccgactataccatggmttgggtccgtcaggcc

SEQ ID NO: 28 - DNA, 81 bp, Artificial sequence
  Mutagenic oligonucleotide
ctggaatgggttgcagatgttaatscaaacagtgstgstkccatckmtaaccagsstkytrmagstcgtttcactctgagt

SEQ ID NO: 29 - DNA, 60 bp, Artificial sequence
  Mutagenic oligonucleotide
tattattgtgctcgtaacctggstccctctkytkmtkytgmtkmttggggtcaaggaacc

SEQ ID NO: 30 - DNA, 66 bp, Artificial sequence
  Mutagenic oligonucleotide
gtcaccatcacctgcargkcckccsaagamrttkccrttgstrttkcctggtatcaacagaaacca

SEQ ID NO: 31 - DNA, 51 bp, Artificial sequence
  Mutagenic oligonucleotide
aaactactgatttackcckcckcctwcargtwcascggagtcccttctcgc

SEQ ID NO: 32 - DNA, 57 bp, Artificial sequence
  Mutagenic oligonucleotide
gcaacttattactgtsaasaatwctwcrtttwcscatwcasctttggacagggtacc

SEQ ID NO: 33 - DNA, 54 bp, Artificial sequence
  Mutagenic oligonucleotide
gcagcttctggcttcascttcascgamtwcascmtggamtgggtccgtcaggcc

SEQ ID NO: 34 - DNA, 84 bp, Artificial sequence
  Mutagenic oligonucleotide
ggcctggaatgggttgcagamrttracscarackccgstgstkccrtttwcracsaaargtwcarggstcgtttcactctgagt

SEQ ID NO: 35 - DNA, 60 bp, Artificial sequence
  Mutagenic oligonucleotide
tattattgtgctcgtracmtcgstscakcctwctwctwcgamtwctggggtcaaggaacc

SEQ ID NO: 36 - DNA, 36 bp, Artificial sequence
  Mutagenic oligonucleotide
gcagcttctggcttcacctttaacgactataccatg

SEQ ID NO: 37 - DNA, 36 bp, Artificial sequence
  Mutagenic oligonucleotide
ctggaatgggttgcagacgttaatcctaacagtggc

SEQ ID NO: 38 - DNA, 36 bp, Artificial sequence
  Mutagenic oligonucleotide
tattattgtgctcgtaacctgggaccctctttctac

(19) United States
(12) Patent Application Publication
(10) Pub. No.: US 2003/0180714 A1
(43) Pub. Date: Sep. 25, 2003

(54) Shotgun scanning

(75) Inventors: Sachdev S. Sidhu, San Francisco, CA (US); Gregory A. Weiss, Irvine, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 09/738,937

(22) Filed: Dec. 14, 2000

Related U.S. Application Data
(60) Provisional application No. 60/170,982, filed on Dec. 15, 1999.

(51) Int. Cl. C12P 21/06; C12N 15/86

(52) U.S. Cl. 435/5; 435/7.1; 435/69.1; 435/456; 435/325

(57) ABSTRACT

A combinatorial method that uses statistics and DNA sequence analysis rapidly assesses the functional and structural importance of individual protein side chains to binding interactions. This general method, termed “shotgun scanning”, enables the rapid mapping of functional protein and peptide epitopes and is suitable for high throughput proteomics.

### FIELD OF THE INVENTION

The invention relates to a method for determining which amino acid residues in a binding protein interact with a ligand capable of binding to the protein. More specifically, the invention is a method of scanning a protein to determine important binding residues in the binding interaction between the protein and the ligand. The invention can be used to prepare libraries, for example phage display libraries, as well as the vectors and host cells containing the vectors.

### DISCUSSION OF THE BACKGROUND

Bacteriophage (phage) display is a technique by which variant polypeptides are displayed as fusion proteins to the coat protein on the surface of bacteriophage particles (Scott, J. K. and Smith, G. P. (1990) Science 249: 386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409; 5,403,484; 5,571,689; 5,663,143.

Typically, variant polypeptides are fused to a gene III protein, which is displayed at one end of the viron. Alternatively, the variant polypeptides may be fused to the gene VIII protein, which is the major coat protein of the viron. Such polyvalent display libraries are constructed by replacing the phage gene III with a cDNA encoding the foreign sequence fused to the amino terminus of the gene III protein. This can complicate efforts to sort high affinity variants from libraries because of the avidity effect; phage can bind to the target through multiple point attachment. Moreover, because the gene III protein is required for attachment and propagation of phage in the host cell, e.g., E. coli, the fusion protein can dramatically reduce infectivity of the progeny phage particles.

To overcome these difficulties, monovalent phage display was developed in which a protein or peptide sequence is fused to a portion of a gene III protein and expressed at low levels in the presence of wild-type gene III protein so that particles display mostly wild-type gene III protein and one copy or none of the fusion protein (Bass, S. et al. (1990) Proteins, 8:309; Lowman, H. B. and Wells, J. A. (1991) Methods: a Companion to Methods in Enzymology, 3:205). Monovalent display has advantages over polyvalent phage display in that progeny phagemnid particles retain full infectivity. Avidity effects are reduced so that sorting is on the basis of intrinsic ligand affinity, and phagemid vectors, which simplify DNA manipulations, are used. See also U.S. Pat. Nos. 5,750,373 and 5,780,279. Others have also used phagemids to display proteins, particularly antibodies. U.S. Pat. Nos. 5,667,988; 5,759,817; 5,770,356; and 5,658,727.

A two-step approach has been used to select high affinity ligands from peptide libraries displayed on M13 phage. Low affinity leads were first selected from naive, polyvalent libraries displayed on the major coat protein (protein VIII). The low affinity selectants were subsequently transferred to the gene III minor coat protein and matured to high affinity in a monovalent format. Unfortunately, extension of this methodology from peptides to proteins has been difficult. Display levels on protein VIII vary with fusion length and sequence. Increasing fusion size generally decreases display. Thus, while monovalent phage display has been used to affinity mature many different proteins, polyvalent display on protein VIII has not been applicable to most protein scaffolds.

Although most phage display methods have used filamentous phage, lambdoid phage display systems (WO 95/34683; U.S. Pat. No. 5,627,024), T4 phage display systems (Ren, Z-J. et al. (1998) Gene 215:439; Zhu, Z. (1997) CAN 33:534; Jiang, J. et al. (1997) can 128:44380; Ren, Z-J. et al. (1997) CAN 127:215644; Ren, Z-J. (1996) Protein Sci. 5:1833; Efimov, V. P. et al. (1995) Virus Genes 10:173) and T7 phage display systems (Smith, G. P. and Scott, J. K. (1993) Methods in Enzymology, 217, 228-257; U.S. Pat. No. 5,766,905) are also known.

Many other improvements and variations of the basic phage display concept have now been developed. These improvements enhance the ability of display systems to screen peptide libraries for binding to selected target molecules and to display functional proteins with the potential of screening these proteins for desired properties. Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphlylococcus aureus protein A as an affinity tag has also been reported (Li et al. (1998) Mol Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538; 5,432,018; and WO 98/15833.

Methods of generating peptide libraries and screening these libraries are also disclosed in U.S. Pat. Nos. 5,723,286; 5,432,018; 5,580,717; 5,427,908; and 5,498,530. See also U.S. Pat. Nos. 5,770,434; 5,734,018; 5,698,426; 5,763,192; and 5,723,323.

Methods which alter the infectivity of phage are also known. WO 95/34648 and U.S. Pat. No. 5,516,637 describe a method of displaying a target protein as a fusion protein with a pilin protein of a host cell, where the pilin protein is preferably a receptor for a display phage. U.S. Pat. No. 5,712,089 describes infecting a bacteria with a phagemid expressing a ligand and then superinfecting the bacteria with helper phage containing wild type protein III but not a gene encoding protein III followed by addition of a protein HI-second ligand where the second ligand binds to the first ligand displayed on the phage produced. See also WO 96/22393. A selectively infective phage system using non-infectious phage and an infectivity mediating complex is also known (U.S. Pat. No. 5,514,548).

Phage systems displaying a ligand have also been used to detect the presence of a polypeptide binding to the ligand in a sample (WO/9744491), and in an animal (U.S. Pat. No. 5,622,699). Methods of gene therapy (WO 98/05344) and drug delivery (WO 97/12048) have also been proposed using phage which selectively bind to the surface of a mammalian cell.

Further improvements have enabled the phage display system to express antibodies and antibody fragments on a bacteriophage surface, allowing for selection of specific properties, i.e., binding with specific ligands (EP 844306; U.S. Pat. Nos. 5,702,892; 5,658,727) and recombination of antibody polypeptide chains (WO 97/09436). A method to generate antibodies recognizing specific peptide—MHC complexes has also been developed (WO 97/02342). See also U.S. Pat. Nos. 5,723,287; 5,565,332; and 5,733,743.

U.S. Pat. No. 5,534,257 describes an expression system in which foreign epitopes up to about 30 residues are incorporated into a capsid protein of a MS-2 phage. This phage is able to express the chimeric protein in a suitable bacterial host to yield empty phage particles free of phage RNA and other nucleic acid contaminants. The empty phage are useful as vaccines.

Gregoret, L. M. and Sauer, R. T., 1993, Proc. Natl. Acad. Sci. USA 90:4246-4250 describe the binomial mutagenesis of eleven amino acids in the helix-turn-helix of X repressor using a combinatorial method. For mutagenesis, a double-stranded cassette was synthesized and each strand was made so that at 11 mutated positions, a 1:1 mixture of bases was used that would create either the codon for the wild-type amino acid or alanine. Pairwise interactions were evaluated. This approach uses a single library to provide information on several residue positions. However, the technique is limited to proteins that can be genetically selected in E. coli, and thus is not applicable to most mammalian proteins. Furthermore, in vivo selections cannot distinguish between structural and functional perturbations to the protein.

Methods of transforming cells to introduce new DNA are well known in molecular biology and modern genetic engineering. Early methods involved chemical treatment of bacteria with solutions of metal ions, generally calcium chloride, followed by heating to produce competent bacteria capable of functioning as recipient bacteria and able to take up heterologous DNA derived from a variety of sources. These early protocols provided transformation yields of about 10⁵-10⁶ transformed colonies per μgram of plasmid DNA. Subsequent improvements using different cations, longer treatment times and other chemical agents have allowed improvements in transformation efficiency of up to about 10⁸ colonies/g gram of DNA. Sambrook et al., Molecular Cloning: A Laboratory Manual, 2nd edition, (1989) Cold Spring Harbor Laboratory Press, Cold Spring Harbor, N.Y., page 1.74.

Cells can also be transformed using high-voltage electroporation. Electroporation is suitable introduce DNA into eukaryotic cells (e.g. animal cells, plant cells, etc.) as well as bacteria, e.g., E. coli. Sambrook et al., ibid, pages 1.75, 16.54-16.55. Different cell types require different conditions for optimal electroporation and preliminary experiments are generally conducted to find acceptable levels of expression or transformation. For mammalian cells, voltages of 250-750 V/cm result in 20-50% cell survival. An electric pulse length of 20-100 ms at a temperature ranging from room temperature to 0° C. and below using a DNA concentration of 1-40 μgram/mL are typical parameters. Transfection efficiency is reported to be higher using linear DNA and when the cells are suspended in buffered salt solutions than when suspended in nonionic solutions. Sambrook et al., above, pages 16.54-16.55. See also Dower et al., 1988, Nucleic Acids Research, 16:6127-6145; U.S. Pat. Nos. 4,910,140; 5,186,800; and 4,849,355. Additional references teaching various aspects of electroporation and/or transformation include U.S. Pat. Nos. 5,173,158; 5,098,843; 5,422,272; 5,232,856; 5,283,194; 5,128,257; 5,124,259 and 4,956,288.

An important emerging use of cell transformations, including electroporation, is the preparation of peptide and protein variant libraries. In these applications, a replicable transcription or expression vector, for example a plasmid, phage or phagemid, is reacted with a restriction enzyme to open the vector DNA, desired coding DNA is ligated into the vector to form a library of vectors each encoding a different variant, and cells are transformed with the library of transformation vectors in order to prepare a library of polypeptide variants differing in amino acid sequence at one or more residues. The library of peptides can then be selectively panned for peptides which have or do not have particular properties. A common property is the ability of the variant peptides to bind to a cell surface receptor, an antibody, a ligand or other binding partner, which may be bound to a solid support. Variants may also be selected for their ability to catalyze specific reactions, to inhibit reactions, to inhibit enzymes, etc.

In one application, bacteriophage (phage), such as filamentous phage, are used to create phage display libraries by transforming host cells with phage vector DNA encoding a library of peptide variants. J. K. Scott and G. P. Smith, Science, (1990), 249:386-390. Phagemid vectors may also be used for phage display. Lowman and Wells, 1991, Methods: A Companion to Methods in Enzymology, 3:205-216. The preparation of phage and phagemid display libraries of peptides and proteins, e.g. antibodies, is now well known in the art. These methods generally require transforming cells with phage or phagemid vector DNA to propagate the libraries as phage particles having one or more copies of the variant peptides or proteins displayed on the surface of the phage particles. See, for example, Barbas et al., Proc. Natl. Acad. Sci., USA, (1991), 88:7978-7982; Marks et al., J. Mol. Biol., (1991), 222:581-597; Hoogenboom and Winter, J. Mol. Biol., (1992), 227:381-388; Barbas et al., Proc. Natl. Acad. Sci., USA, (1992), 89:4457-4461; Griffiths et al., EMBO Journal, (1994), 13:3245-3260; de Kruif et al., J. Mol. Biol., (1995), 248:97-105; Bonnycastle et al., J. Mol. Biol., (1996), 258:747-762; and Vaughan et al., Nature Biotechnology (1996), 14:309-314. The library DNA is prepared using restriction and ligation enzymes in one of several well known mutagenesis procedures, for example, cassette mutagenesis or oligonucleotide-mediated mutagenesis.

Notwithstanding numerous modifications and improvements in phage technology and in protein engineering in general, a need continues to exist for improved methods of displaying polypeptides as fusion proteins in phage display methods and improved methods of protein engineering.

### SUMMARY OF THE INVENTION

Progress in DNA technologies has outpaced techniques for protein analysis. As a result, the human genome sequence is nearing completion, but the details of many protein-protein interactions are not known. The fine details of receptor-ligand interactions by proteins in the proteome requires specialized techniques, such as X-ray crystallography, which must be adapted for each interaction. This dichotomy reflects a fundamental difference between DNA and peptide biopolymers. While DNA can be readily manipulated without regard for sequence, different protein sequences can produce different three-dimensional structures with highly variable physical properties.

An object of the invention is, therefore, to provide a general method of determining which amino acid positions in a polypeptide play a role in ligand binding to the polypeptide and to provide a general method of indicating the relative importance of a particular residue to the structural integrity or, alternatively, to the functional integrity of the polypeptide.

Although rapid analysis of the proteome requires general methods, the unique properties of individual proteins demand specialized techniques. The present invention is a method of “shotgun scanning”, a general technique for receptor-ligand analysis, which relies primarily upon manipulation of DNA. Use of DNA technologies and library sorting techniques, preferably through phage display, confers at least two advantages. First, shotgun scanning is very rapid, and can be automated. Secondly, the technique can be readily adapted to many receptor-ligand interactions.

One embodiment of the invention is a library of fusion genes encoding a plurality of fusion proteins, where the fusion proteins comprise a polypeptide portion fused to at least a portion of a phage coat protein, the polypeptide portions of the fusion proteins differ at a predetermined number of amino acid positions, and the fusion genes encode at most eight different amino acids at each predetermined amino acid position.

Another embodiment of the invention is a library of expression vectors containing fusion genes encoding a plurality of fusion proteins, wherein the fusion proteins comprise a polypeptide portion fused to at least a portion of a phage coat protein, the polypeptide portions of the fusion proteins differ at a predetermined number of amino acid positions, and the fusion genes encode at most eight different amino acids at each predetermined amino acid position.

A further embodiment is library of phage or phagemid particles containing fusion genes encoding a plurality of fusion proteins, wherein the fusion proteins comprise a polypeptide portion fused to at least a portion of a phage coat protein, the polypeptide portion of the fusion proteins differs at a predetermined number of amino acid positions, and the fusion genes encode at most eight different amino acids at each predetermined amino acid position.

Preferably, the fusion genes encode a wild type amino acid which naturally occurs in the polypeptide, a scanning amino acid (e.g., a single scanning amino acid or a homolog) and 2, 3, 4, 5 or 6 non-wild type, non-scanning amino acids or a stop codon (for example, a suppressible stop codon such as amber or ochre) at each predetermined amino acid position. The non-wild type, non-scanning amino acids may be any of the remaining naturally occurring amino acids. The fusion genes may encode a wild type amino acid and a scanning amino acid at one or more predetermined amino acid positions. Alternatively, the fusion genes may encode only a wild type amino acid and a scanning amino acid at each predetermined amino acid position. The scanning amino acid may be alanine, cysteine, isoleucine, phenylalanine, or any of the other well known naturally occurring amino acids. The fusion genes preferably encode alanine as the scanning amino acid at each predetermined amino acid position. The predetermined number may be in the range 2-60, preferably 5-40, more preferably 5-35 or 10-50 amino acid positions in the polypeptide.

In another embodiment, the invention provides a method for constructing the library of phage or phagemid particles described above, where the fusion genes encode a wild type amino acid, a scanning amino acid and up to six non-wild type, non-scanning amino acids at each predetermined amino acid position and the particles display the fusion proteins on the surface thereof. The library of particles is then contacted with a target molecule so that at least a portion of the particles bind to the target molecule; and the particles that bind are separated from those that do not bind. One may determine the ratio or frequency of wild-type to scanning amino acids at one or more, preferably all, of the predetermined positions for at least a portion of polypeptides on the particles which bind or which do not bind. Generally, the polypeptide and target molecule are selected from the group of polypeptide/target molecule pairs consisting of ligand/receptor, receptor/ligand, ligand/antibody, antibody/ligand, where the term ligand includes both biopolymers and small molecules.

In another embodiment, the invention is directed to a method for producing a product polypeptide by (1) culturing a host cell transformed with a replicable expression vector, the replicable expression vector comprising DNA encoding a product polypeptide operably linked to a control sequence capable of effecting expression of the product polypeptide in the host cell; where the DNA encoding the product polypeptide has been obtained by a method including the steps of:

(a) constructing a library of expression vectors containing fusion genes encoding a plurality of fusion proteins, where the fusion proteins comprise a polypeptide portion fused to at least a portion of a phage coat protein, the polypeptide portions of the fusion proteins differ at a predetermined number of amino acid positions, and the fusion genes encode at most eight different amino acids at each predetermined amino acid position;

(b) transforming suitable host cells with the library of expression vectors;

(c) culturing the transformed host cells under conditions suitable for forming recombinant phage or phagemid particles displaying variant fusion proteins on the surface thereof;

(d) contacting the recombinant particles with a target molecule so that at least a portion of the particles bind to the target molecule;

(e) separating particles that bind to the target molecule from those that do not bind;

(f) selecting one of the variant as the product polypeptide and cloning DNA encoding the product polypeptide into the replicable expression vector; and (2) recovering the expressed product polypeptide. Optionally, the variant selected may be mutated using well known techniques such as cassette mutagenesis or oligonucleotide mutagenesis to form a mutated variant which may then be selected and produced as the product polypeptide.

In a further embodiments, the invention is directed to a method of determining the contribution of individual amino acid side chains to the binding of a polypeptide to a ligand therefor, including the steps of

constructing a library of phage or phagemid particles as described herein;

contacting the library of particles with a target molecule so that at least a portion of the particles bind to the target molecule; and

separating the particles that bind from those that do not bind. When a wild type amino acid and a scanning amino acid are encoded at each predetermined amino acid position the method of the invention may further include a step of determining the ratio of wild-type:scanning amino acid at one or more, preferably all, of the predetermined positions for at least a portion of polypeptides on the particles which bind or which do not bind.

This and other objects which will become apparent in the course of the following descriptions of exemplary embodiments have been achieved by the present method and other embodiments of the invention.

### BRIEF DESCRIPTION OF THE DRAWINGS

FIG. 1 shows the results of shotgun scanning human growth hormone (hGH), with selection for human growth hormone binding protein (hGHbp, dark, right bar of each pair) or anti-hGH antibody (light, left bar of each pair), for 19 mutated hGH residues α-axis). Fraction wild-type (y-axis) was calculated by Σn_{wild-type}/Σ(n_{wild-type}+nₐₗₐₙᵢₙₑ) from the sequences of 330 hGHbp selected or 175 anti-hGH antibody selected clones. Error bars represent 95% confidence levels.

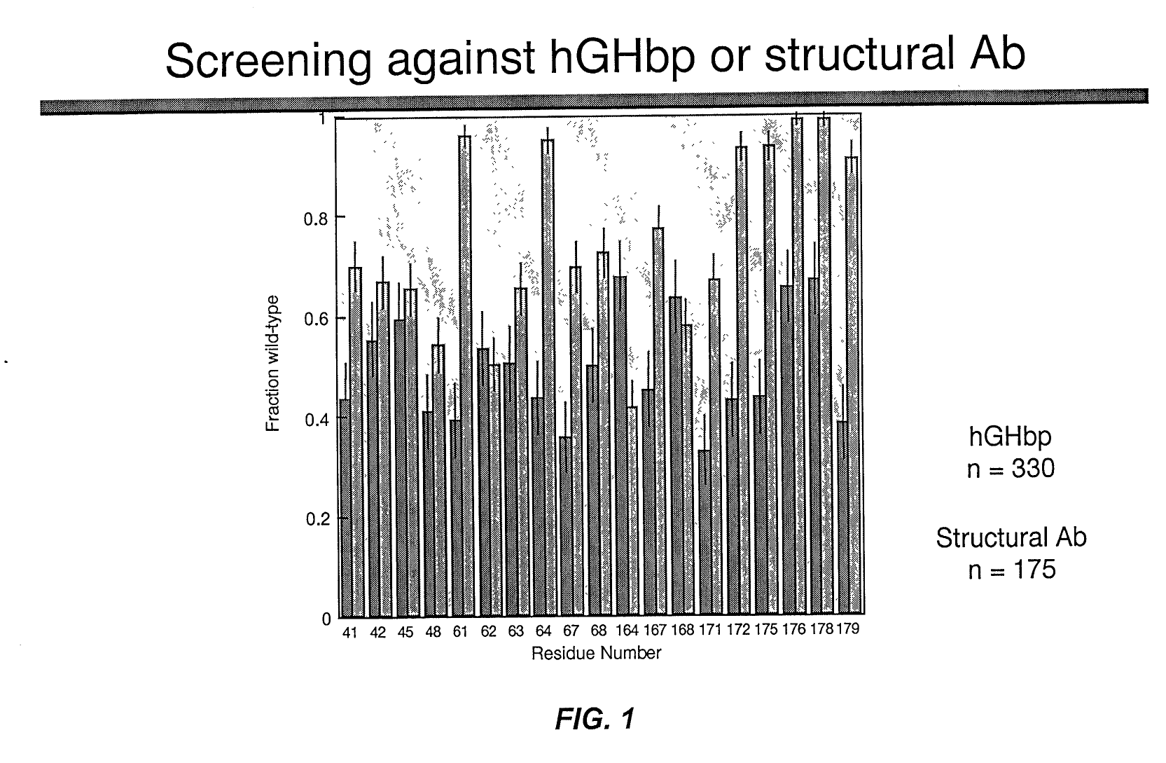

FIG. 2 shows the shotgun scanning α-axis) versus alanine mutagenesis of individual residues (y-axis). Alanine mutagenesis data, shown here as the ΔΔG upon binding for each hGH mutant was measured according to Cunningham and Wells, 1993, J. Mol. Biol. 234:554.

### DETAILED DESCRIPTION OF THE PREFERRED EMBODIMENTS

### DEFINITIONS

The term “affinity purification” means the purification of a molecule based on a specific attraction or binding of the molecule to a chemical or binding partner to form a combination or complex which allows the molecule to be separated from impurities while remaining bound or attracted to the partner moiety.

“Alanine scanning” is a site directed mutagenesis method of replacing amino acid residues in a polypeptide with alanine to scan the polypeptide for residues involved in an interaction of interest (Clackson and Wells, 1995, Science 267:383). Alanine scanning has been particularly successful in systematically mapping functional binding epitopes (Cunningham and Wells, 1989, Science 244:1081; Matthews, 1996, FASEB J. 10:35; Wells, 1991, Meth. Enzymol. 202:390).

The term “antibody” is used in the broadest sense and specifically covers single monoclonal antibodies (including agonist and antagonist antibodies), antibody compositions with polyepitopic specificity, affinity matured antibodies, humanized antibodies, chimeric antibodies, as well as antibody fragments (e.g., Fab, F(ab′)₂, scFv and Fv), so long as they exhibit the desired biological activity. An affinity matured antibody will typically have its binding affinity increased above that of the isolated or natural antibody or fragment thereof by from 2 to 500 fold. Preferred affinity matured antibodies will have nanomolar or even picomolar affinities to the receptor antigen. Affinity matured antibodies are produced by procedures known in the art. Marks, J. D. et al. Bio/Technology 10:779-783 (1992) describes affinity maturation by VH and VL domain shuffling. Random mutagenesis of CDR and/or framework residues is described by: Barbas, C. F. et al. Proc Nat. Acad. Sci, USA 91:3809-3813 (1994), Schier, R. et al. Gene 169:147-155 (1995), Yelton, D. E. et al., J. Immunol. 155:1994-2004 (1995), Jackson, J. R. et al., J. Immunol. 154(7):3310-9 (1995), and Hawkins, R. E. et al, J. Mol. Biol. 226:889-896 (1992). Humanized antibodies are known. Jones et al., Nature, 321:522-525 (1986); Reichmann et al., Nature, 332:323-329 (1988); and Presta, Curr. Op. Struct. Biol., 2:593-596 (1992)).

An “Fv” fragment is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other, chemical couplings of antibody fragments are also known.

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The expression “linear antibodies” refers to the antibodies described in Zapata et al. Protein Eng. 8(10):1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

“Cell,” “cell line,” and “cell culture” are used interchangeably herein and such designations include all progeny of a cell or cell line. Thus, for example, terms like “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The terms “competent cells” and “electoporation competent cells” mean cells which are in a state of competence and able to take up DNAs from a variety of sources. The state may be transient or permanent. Electroporation competent cells are able to take up DNA during electroporation.

“Control sequences” when referring to expression means DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

The term “coat protein” means a protein, at least a portion of which is present on the surface of the virus particle. From a functional perspective, a coat protein is any protein which associates with a virus particle during the viral assembly process in a host cell, and remains associated with the assembled virus until it infects another host cell. The coat protein may be the major coat protein or may be a minor coat protein. A “major” coat protein is a coat protein which is present in the viral coat at 10 copies of the protein or more. A major coat protein may be present in tens, hundreds or even thousands of copies per virion.

The terms “electroporation” and “electroporating” mean a process in which foreign matter (protein, nucleic acid, etc.) is introduced into a cell by applying a voltage to the cell under conditions sufficient to allow uptake of the foreign matter into the cell. The foreign matter is typically DNA.

An “F factor” or “F′ episome” is a DNA which, when present in a cell, allows bacteriophage to infect the cell. The episome may contain other genes, for example selection genes, marker genes, etc. Common F′ episomes are found in well known E. coli strains including CJ236, CSH18, DHSalphaF′, JM101 (same as in JM103, JM105, JM107, JM109, JM110), KS1000, XL1-BLUE and 71-18. These strains and the episomes contained therein are commercially available (New England Biolabs) and many have been deposited in recognized depositories such as ATCC in Manassas, Va.

A “fusion protein” is a polypeptide having two portions covalently linked together, where each of the portions is a polypeptide having a different property. The property may be a biological property, such as activity in vitro or in vivo. The property may also be a simple chemical or physical property, such as binding to a target molecule, catalysis of a reaction, etc. The two portions may be linked directly by a single peptide bond or through a peptide linker containing one or more amino acid residues. Generally, the two portions and the linker will be in reading frame with each other.

“Heterologous DNA” is any DNA that is introduced into a host cell. The DNA may be derived from a variety of sources including genomic DNA, cDNA, synthetic DNA and fusions or combinations of these. The DNA may include DNA from the same cell or cell type as the host or recipient cell or DNA from a different cell type, for example, from a mammal or plant. The DNA may, optionally, include selection genes, for example, antibiotic resistance genes, temperature resistance genes, etc.

“Ligation” is the process of forming phosphodiester bonds between two nucleic acid fragments. For ligation of the two fragments, the ends of the fragments must be compatible with each other. In some cases, the ends will be directly compatible after endonuclease digestion. However, it may be necessary first to convert the staggered ends commonly produced after endonuclease digestion to blunt ends to make them compatible for ligation. For blunting the ends, the DNA is treated in a suitable buffer for at least 15 minutes at 15° C. with about 10 units of the Klenow fragment of DNA polymerase I or T4 DNA polymerase in the presence of the four deoxyribonucleotide triphosphates. The DNA is then purified by phenol-chloroform extraction and ethanol precipitation. The DNA fragments that are to be ligated together are put in solution in about equimolar amounts. The solution will also contain ATP, ligase buffer, and a ligase such as T4 DNA ligase at about 10 units per 0.5 μg of DNA. If the DNA is to be ligated into a vector, the vector is first linearized by digestion with the appropriate restriction endonuclease(s). The linearized fragment is then treated with bacterial alkaline phosphatase or calf intestinal phosphatase to prevent self-ligation during the ligation step.

“Operably linked” when referring to nucleic acids means that the nucleic acids are placed in a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adapters or linkers are used in accord with conventional practice.

“Phage display” is a technique by which variant polypeptides are displayed as fusion proteins to a coat protein on the surface of phage, e.g. filamentous phage, particles. A utility of phage display lies in the fact that large libraries of randomized protein variants can be rapidly and efficiently sorted for those sequences that bind to a target molecule with high affinity. Display of peptides and proteins libraries on phage has been used for screening millions of polypeptides for ones with specific binding properties. Polyvalent phage display methods have been used for displaying small random peptides and small proteins through fusions to either gene III or gene VIII of filamentous phage. Wells and Lowman, Curr. Opin. Struct. Biol., 1992, 3:355-362 and references cited therein. In monovalent phage display, a protein or peptide library is fused to a gene III or a portion thereof and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. Lowman and Wells, Methods: A companion to Methods in Enzymology, 1991, 3:205-216.

A “phagemid” is a plasmid vector having a bacterial origin of replication, e.g., ColE1, and a copy of an intergenic region of a bacteriophage. The phagemid may be based on any known bacteriophage, including filamentous bacteriophage and lambdoid bacteriophage. The plasmid will also generally contain a selectable marker for antibiotic resistance. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid may form infectious or non-infectious phage particles. This term includes phagemids which contain a phage coat protein gene or fragment thereof linked to a heterologous polypeptide gene as a gene fusion such that the heterologous polypeptide is displayed on the surface of the phage particle. Sambrook et al., above, 4.17.

The term “phage vector” means a double stranded replicative form of a bacteriophage containing a heterologous gene and capable of replication. The phage vector has a phage origin of replication allowing phage replication and phage particle formation. The phage is preferably a filamentous bacteriophage, such as an M13, fl, fd, Pf3 phage or a derivative thereof, or a lambdoid phage, such as lambda, 21, phi80, phi81, 82, 424, 434, etc., or a derivative thereof.

A “predetermined” number of amino acid positions is simply the number amino acid positions which are scanned in a polypeptide. The predetermined number may range from 1 to the total number of amino acid residues in the polypeptide. Usually, the predetermined number will be more than one and will range from 2 to about 60, preferably 5 to about 40, more preferably 5 to about 35 amino acid positions. The number of predetermined positions may also be 3, 4, 6, 7, 8, 9, 10, 11, 12, 13, 14, 15, 16, 17, 18, 19, 20, etc. The predetermined positions may be scanned using a single library or multiple libraries as practicable.

“Preparation” of DNA from cells means isolating the plasmid DNA from a culture of the host cells. Commonly used methods for DNA preparation are the large- and small-scale plasmid preparations described in sections 1.25-1.33 of Sambrook et al., supra. After preparation of the DNA, it can be purified by methods well known in the art such as that described in section 1.40 of Sambrook et al., supra.

“Oligonucleotides” are short-length, single- or double-stranded polydeoxynucleotides that are chemically synthesized by known methods (such as phosphotriester, phosphite, or phosphoramidite chemistry, using solid-phase techniques such as described in EP 266,032 published May 4, 1988, or via deoxynucleoside H-phosphonate intermediates as described by Froehler et al., Nucl. Acids Res., 14:5399-5407 (1986)). Further methods include the polymerase chain reaction defined below and other autoprimer methods and oligonucleotide syntheses on solid supports. All of these methods are described in Engels et al., Agnew. Chem. Int. Ed. Engl., 28:716-734 (1989). These methods are used if the entire nucleic acid sequence of the gene is known, or the sequence of the nucleic acid complementary to the coding strand is available. Alternatively, if the target amino acid sequence is known, one may infer potential nucleic acid sequences using known and preferred coding residues for each amino acid residue. The oligonucleotides are then purified on polyacrylamide gels.

“Polymerase chain reaction” or “PCR” refers to a procedure or technique in which minute amounts of a specific piece of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template, to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51:263 (1987); Erlich, ed., PCR Technology, (Stockton Press, NY, 1989). As used herein, PCR is considered to be one, but not the only, example of a nucleic acid polymerase reaction method far amplifying a nucleic acid test sample comprising the use of a known nucleic acid as a primer and a nucleic acid polymerase to amplify or generate a specific piece of nucleic acid.

DNA is “purified” when the DNA is separated from non-nucleic acid impurities. The impurities may be polar, non-polar, ionic, etc.

“Recovery” or “isolation” of a given fragment of DNA from a restriction digest means separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. This procedure is known generally. For example, see Lawn et al., Nucleic Acids Res., 9:6103-6114 (1981), and Goeddel et al., Nucleic Acids Res., 8:4057 (1980).

A “small molecule” is a molecule having a molecular weight of about 600 g/mole or less.

A chemical group or species having a “specific binding affinity for DNA” means a molecule or portion thereof which forms a non-covalent bond with DNA which is stronger than the bonds formed with other cellular components including proteins, salts, and lipids.

A “transcription regulatory element” will contain one or more of the following components: an enhancer element, a promoter, an operator sequence, a repressor gene, and a transcription termination sequence. These components are well known in the art. U.S. Pat. No. 5,667,780.

A “transformant” is a cell which has taken up and maintained DNA as evidenced by the expression of a phenotype associated with the DNA (e.g., antibiotic resistance conferred by a protein encoded by the DNA).

“Transformation” means a process whereby a cell takes up DNA and becomes a “transformant”. The DNA uptake may be permanent or transient.

A “variant” of a starting polypeptide, such as a fusion protein or a heterologous polypeptide (heterologous to a phage), is a polypeptide that 1) has an amino acid sequence different from that of the starting polypeptide and 2) was derived from the starting polypeptide through either natural or artificial (manmade) mutagenesis. Such variants include, for example, deletions from, and/or insertions into and/or substitutions of, residues within the amino acid sequence of the polypeptide of interest. Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites. Methods for generating amino acid sequence variants of polypeptides are described in U.S. Pat. No. 5,534,615, expressly incorporated herein by reference.

Generally, a variant coat protein will possess at least 20% or 40% sequence identity and Lip to 70% or 85% sequence identity, more preferably up to 95% or 99.9% sequence identity, with the wild type coat protein. Percentage sequence identity is determined, for example, by the Fitch et al., Proc. Natl. Acad. Sci. USA 80:1382-1386 (1983), version of the algorithm described by Needleman et al., J. Mol. Biol. 48:443-453 (1970), after aligning the sequences to provide for maximum homology. Amino acid sequence variants of a polypeptide are prepared by introducing appropriate nucleotide changes into DNA encoding the polypeptide, or by peptide synthesis. An “altered residue” is a deletion, insertion or substitution of an amino acid residue relative to a reference amino acid sequence, such as a wild type sequence.

A “functional” mutant or variant is one which exhibits a detectable activity or function which is also detectably exhibited by the wild type protein. For example, a “functional” mutant or variant of the major coat protein is one which is stably incorporated into the phage coat at levels which can be experimentally detected. Preferably, the phage coat incorporation can be detected in a range of about 1 fusion per 1000 virus particles up to about 1000 fusions per virus particle.

A “wild type” sequence or the sequence of a “wild type” polypeptide is the reference sequence from which variant polypeptides are derived through the introduction of mutations. In general, the “wild type” sequence for a given protein is the sequence that is most common in nature. Similarly, a “wild type” gene sequence is the sequence for that gene which is most commonly found in nature. Mutations may be introduced into a “wild type” gene (and thus the protein it encodes) either through natural processes or through man induced means. The products of such processes are “variant” or “mutant” forms of the original “wild type” protein or gene.

### DETAILED DESCRIPTION OF THE PREFERRED EMBODIMENTS

The method of the invention, termed “shotgun scanning” is a general combinatorial method for mapping structural and functional epitopes of proteins. Combinatorial protein libraries are constructed in which residues are preferably allowed to vary only as the wild-type or as a scanning amino acid, for example, alanine. In another aspect of the invention, the degeneracy of the genetic code necessitates two or more, e.g. 2-6, other amino acid substitutions or, optionally a stop codon, for some residues. Because the diversity is limited to only a few possibilities at each position, current library construction technologies allow the simultaneous mutation of a plurality, generally 1 to about 60, more preferably 1 to about 40, even more preferably about 5 to about 25 or to about 35, of positions with reasonable probability of complete coverage. The library pool may be displayed on phage particles, for example filamentous phage particles, and in vitro selections are used to isolate members retaining binding for target ligands, which are preferably immobilized on a solid support. Selected clones are sequenced, and the occurrence of wild-type or scanning amino acid at each position is tabulated. Depending on the nature of the selected interaction, this information can be used to assess the contribution of each side chain to protein structure and/or function. Shotgun scanning is extremely rapid and simple. Many side chains are analyzed simultaneously using highly optimized DNA sequencing techniques, and the need for substantial protein purification and analysis is circumvented. This technique is applicable to essentially any protein that can be displayed on a bacteriophage.

The method of the invention has several advantages over conventional saturation mutagenesis methods to generate variant polypeptides in which any of the naturally occurring amino acids may be present at one or more predetermined sites on the polypeptide. Traditionally, protein engineering has used saturation mutagenesis to create a library of variants or mutants and then checked the binding or activity of each variant/mutant to determine the effect of that specific variant/mutant on the binding or activity of the protein being studied. No selection process is used in this type of analysis, rather each variant/mutant is studied individually. This process is labor intensive, time consuming and not readily adapted to high throughput applications.

Alternatively, saturation mutagenesis has been combined with a selection process, for example using binding affinity between the studied polypeptide and a binding partner therefore Conventional phage display methods are an example of this approach. Very large libraries of polypeptide variants are generated, screened or panned for binding to a target in one or more rounds of selection, and then a small subset of selectants are sequenced and further analyzed. Although this method is faster than earlier methods, analysis of only a small subset of selectants necessarily results in loss of information. Limiting the number of mutation sites to limit the loss of information is also unsatisfactory since this is more labor intensive and requires iterative rounds of mutation to fully analyze the binding interactions of ligand/receptor pairs. The method of the invention allows for the simultaneous evaluation of the importance of a plurality of amino acid positions to the binding and/or interaction of a polypeptide of interest with a binding partner for the polypeptide. The binding partner may be any ligand for the polypeptide of interest, for example, another polypeptide or protein, such as a cell surface receptor, ligand or antibody, or may be a nucleic acid (e.g., DNA or RNA), small organic molecule ligand or binding target (e.g., drug, pharmaceutical, inhibitor, agonist, blocker, etc.) of the polypeptide of interest, including fragments thereof. For example, the shotgun scanning method of the invention can be used to evaluate the importance of a group of amino acid residues in a binding pocket of a protein or in an active site of an enzyme to the binding of the protein or enzyme to a substrate, agonist, antagonist, inhibitor, ligand, etc.

In general, the method of the invention provides a method for the systematic analysis of the structure and function of polypeptides by identifying unknown active domains and individual amino acid residues within these domains which influence the activity of the polypeptide with a target molecule or with a binding partner molecule. These unknown active domains may comprise a single contiguous domain or may comprise at least two discontinuous domains in the primary amino acid sequence of a polypeptide. Indeed, the shotgun scanning method of the invention is useful for any of the uses that are identified for conventional amino acid scanning technologies. See U.S. Pat. Nos. 5,580,723; 5,766,854; 5,834,250.

When the polypeptide encoded by the first gene is an antibody, the method of the invention can be used to scan the antibody for amino acid residues which are important to binding to an epitope. For example, the complementarity determining regions (CDRs) and/or the framework portions of the variable regions and/or the Fc constant regions may be scanned to determine the relative importance of each residue in these regions to the binding of the antibody to an antigen or target or to other functions of the antibody, for example binding to clearance receptors, complement fixation, cell killing, etc. In an example of this embodiment, shotgun scanning is useful in affinity maturing an antibody. Any antibody, including murine, human, chimeric (for example humanized), and phage display generated antibodies may be scanned with the method of the invention.

The method of the invention may also be used to perform an epitope analysis on the ligand which binds to an antibody. The ligand may be shotgun scanned by generating a library of fusion proteins and expressing the fusion proteins on the surface of phage or phagemid particles using phage display techniques as described herein. Analysis of the ratio of wild-type residues to scanning residues at predetermined positions on the ligand provides information about the contribution of the scanned positions to the binding of the antibody and ligand. Shotgun scanning, therefore, is a tool in protein engineering and a method of epitope mapping a ligand. In an analogous manner, the binding of a ligand and a cell surface receptor can be analyzed. The binding region on the ligand and on the receptor may each be shotgun scanned as a means of mapping the binding residues or the binding patches on each of the respective binding partner proteins.

The shotgun scanning method of the invention may be used as a structural scan of a polypeptide of known amino acid sequence. That is, the method can be used to scan a polypeptide to determine which amino acid residues are important to maintaining the structure of the polypeptide. In this embodiment, residues which perturb the structure of the polypeptide reduce the level of display of the polypeptide as a fusion protein with a phage coat protein on the surface of a phage or phagemid particle. More specifically, if a wild-type residue is replaced with a scanning residue at position Nx of the polypeptide and the resulting variant exhibits poor display relative to the original polypeptide containing the wild-type residue, then position Nx is important to maintaining the three-dimensional structure of the polypeptide. This effect can be determined by finding the frequency of occurrence of the wild-type and/or scanning residues for the Nx position. If the wild-type residue is important to maintaining structure, the wild-type frequency should approach 1.0; if the wild-type residue is not important to maintaining structure, the wild-type frequency should approach 0.0. In practice, frequencies in the entire range from 0.0 to 1.0 are possible for both the wild-type frequency and the scanning residue frequency, since any specific residue may be relatively more or less important to the structure of the polypeptide. Scanning is conducted simultaneously in the method of the invention for multiple positions Nx, where x=1-60, preferably 10-40 or 5-35.

The shotgun scanning method of the invention may also be used as a functional scan of a polypeptide of known amino acid sequence. That is, the method can be used to scan a polypeptide to determine which amino acid residues are important to the function of the polypeptide, for example as reflected in the binding of the polypeptide to a ligand. If the wild-type residue is important to the binding of the polypeptide with the ligand, the wild-type frequency should approach 1.0; if the wild-type residue is not important to the binding, the wild-type frequency should approach 0.0. As described above, frequencies in the entire range from 0.0 to 1.0 are possible for both the wild-type frequency and the scanning residue frequency, since any specific residue may be relatively more or less important to the binding and function of the polypeptide. Scanning is conducted simultaneously in the method of the invention for multiple positions Nx, where x=1-60, preferably 10-40 or 5-35.

The positions Nx to be varied or scanned can be predetermined using known methods of protein engineering which are well known in the art. For example, based on knowledge of the primary structure of the polypeptide, one can create a model of the secondary, tertiary and quaternary (if appropriate) structure of a polypeptide using conventional physical modeling and computer modeling techniques. Such models are generally constructed using physical data such as NMR, IR, and X-ray structure data. Ideally, X-ray crystallographic data will be used to predetermine which residues to scan using the method of the invention. Notwithstanding the preferred use of physical and calculated characterizing data discussed above, one can predetermine the positions to be scanned randomly with knowledge of the primary sequence only. If desired, one can scan the entire polypeptide using a plurality of libraries and scans if the number of predetermined positions exceeds a number which can be varied in a single library. That is, a polypeptide of any size can be entirely scanned using a plurality of libraries and repeatedly scanning through the entire polypeptide.

If desired, a polypeptide can be scanned to determine structurally important residues, for example using an antibody as the target during selection of the phage or phagemid displayed variants, followed by a scan for functionally important residues, for example using a binding ligand or receptor for the polypeptide as the target during selection of the phage or phagemid displayed variants. Other selections are possible and can be used independently or combined with a structural and/or functional scan. Other selections include genetic selection and yeast two- and three-hybrid, using both forward and reverse selections (Warbick, Structure 5: 13-17; Brachmann and Boeke, Curr. Opin. Biotechnol. 8: 561-568).

The method of the invention provides a method for mapping protein functional epitopes by statistically analyzing DNA encoding the polypeptide sequence. For each selection, the sequence data can be used to calculate the wild-type frequency at each position, where wild-type frequency equals Σn_{wild-type}/Σ(n_{wild-type}+nₐₗₐₙᵢₙₑ). The wild-type frequency compares the occurrence of a wild-type side chain relative to alanine, and thus, correlates with a given side chain's contribution to the selected trait (i.e. binding to receptor). The wild-type frequency for a large, favorable contribution to the binding interaction should approach 1.0 (100% enrichment for the wild-type sidechain). The wild-type frequency for a large, negative contribution to binding should approach 0.0, which would result from selection against the wild-type side chain). These calculations may be made manually or using a computer which may be programmed using well known methods. A suitable computer program is “sgcount” described below.

Significant structural and functional information can be obtained by shotgun scanning from a single type of scan. For example, a plurality of different antibodies which bind to a polypeptide may be used as separate targets and the polypeptide to be shotgun scanned by displaying variants of the polypeptide is panned against the immobilized antibodies. A high frequency of a wild-type versus scanning residue at a given specific position of the polypeptide against a plurality of antibody targets indicates that the specific residue is important to maintain the structure of the polypeptide. Conversely, a low frequency indicates a functionally important residue which affects (e.g., may lie in or near) the binding site where the polypeptide contacts the antibody.

In one aspect of the invention, the same amino acid is scanned through the polypeptide or portion of a polypeptide of interest. In this aspect, a limited codon set is used which codes for the wild type amino acid and the same scanning amino acid for each of the positions scanned. Table 1, for example, provides a codon set in which a wild type amino acid and alanine are encoded for each scanned position.

Any of the naturally occurring amino acids may be used as the scanning amino acid. Alanine is generally used since the side chain of this amino acid is not charged and is not sterically large. Shotgun scanning with alanine has all of the advantages of traditional alanine scanning, plus the additional advantages of the present invention. See U.S. Pat. Nos. 5,580,723; 5,766,854; 5,834,250. Leucine is useful for steric scanning to evaluate the effect of a sterically large sidechain in each of the scanned positions. Phenylalanine is useful to scan with a relatively large and aromatic sidechain. Similarly, cysteine shotgun scanning can be used to perturb the polypeptide with additional disulfide crosslinking possibilities and thereby determine the effect of such crosslinks on structure and function of the polypeptide. Glutaric acid or arginine shotgun scanning can be used to screen for perturbation by large charged sidechains. For examples of the codon sets used for these different versions of shotgun scanning see Tables 1 through 6.

In another aspect, the scanning amino acid is a homolog of the wild type amino acid in one or more of the scanned positions. A codon set for homolog shotgun scanning is given in Table B. A library can also be constructed in which amino acids are allowed to vary as only the wild-type or a chemically similar amino acid (ie. a homolog). In this case, the mutations introduce only very subtle changes at a given positions, and such a library can be used to assess how precise the role of a wild-type sidechain's role is in protein structure and/or function. For example, some sidechains may be absolutely required for function, as evidenced by a large effect in an alanine-scan, but the function of the sidechain may not be very precise if it can be replaced by chemically similar side chains, as evidenced by minor effects in a homolog scan. On the other hand, if a sidechain plays a critical and precise role in function, the effects of substituting with either alanine or a homolog may both be expected to be large. Thus, alanine-scanning and homolog-scanning provide different, complementary information about a side chain's role in the structure and function of a protein. The alanine-scan assesses how important it is for a particular side chain to be present, while the homolog-scan assesses how critical the exact chemical nature of the side chain is for correct structure and/or function. Together, the two scans provide a more complete picture of the interface than would be possible with either scan alone.

Protein variants include amino acid substitutions, insertions and deletions. In addition to amino acid substitutions, shotgun scanning of insertions can be used for de novo designed proteins, in which protein features such as surfaces, including loops, sheets, and helices, are added to a protein scaffold. Conversely, protein variants with deletions can be used to examine the contribution of specific regions of protein structures, in the context of deliberately omitted surface features. Thus, insertions allow building up of surface features, possibly or with the desire to gain binding interactions, while deletions can be used to erode a binding surface and dissect binding interactions.

The method of the invention is also well suited for automation and high throughput application. For example, assay plates containing multiple wells (96, 384, etc) can be used to simultaneously scan the desired number of predetermined positions. Wells of the plates are coated with the binding partner of the polypeptide of interest (e.g., receptor or antibody) and the required number of libraries are individually added to the separate wells, one library per well. If the desired scan requires two libraries to scan (i.e., mutate) the predetermined number of positions Nx, then two wells would be used and one library added to each well. After allowing sufficient time for binding, the plates are washed to remove non-binding variants and eluted to remove bound variants. The eluted variants are added to E. coli, which are infected by the eluted phage and grown into colonies. All of the steps described above are routinely accomplished using conventional phage display technology. Automated colony picking machines are then used to identify and pick a representative number (e.g., about 10 to several hundred (about 100 to about 900) or even thousands) of individual colonies and transfer the picked bacteria to an array of culture tubes where the E. coli are grown and expanded. Phage or phagemid particles produced by the infected E. coli using standard phage and phage display culture conditions are then obtained and purified from the cultures and subjected to phage ELISA using automated procedures. See Lowman, H B, 1998, Methods Mol. Biol. 87:249-264. Specifically, robotic manipulators of 96-well ELISA plates can be used to perform all steps of a phage ELISA; this enables high-throughput analysis of hundreds to thousands of clones from binding selections, which may be necessary for shotgun scanning of some protein epitopes. However, for the example described here, only a few hundred clones were sequenced following rounds of phage selection and robust statistical data was obtained.

In one aspect of the invention, it is also possible to mix two or more (a plurality) libraries, for example in one well, and complete the washing, panning, and other steps using the variants of the mixed libraries. This aspect is useful, for example, to scan a pool of protein or peptide variants of a plurality of polypeptides of interest having similar structure or amino acid sequence, such as protein homologs or orthologs. Variants to the homologs or orthologs are prepared and scanned as described herein.

Cells may be transformed by electroporating competent cells in the presence of heterologous DNA, where the DNA has been purified by DNA affinity purification. Preferably, for library construction in bacteria, the DNA is present at a concentration of 25 micrograms/mL or greater. Preferably, the DNA is present at a concentration of about 30 micrograms/mL or greater, more preferably at a concentration of about 70 micrograms/mL or greater and even more preferably at a concentration of about 100 micrograms/mL or greater even up to several hundreds of micrograms/mL. Generally, the method of the invention will utilize DNA concentrations in the range of about 50 to about 500 micrograms/mL. By highly purifying the heterologous DNA, a time constant during electroporation greater than 3.0 milliseconds (ms) is possible even when the DNA concentration is very high, which results in a high transformation efficiency. Over the DNA concentration range of about 50 microgram/mL to about 400 microgram/mL, the use of time constants in the range of about 3.6 to about 4.4 ms is allowed using standard electroporation instruments.

High DNA concentrations may be obtained by highly purifying DNA used to transform the competent cells. The DNA is purified to remove contaminants which increase the conductance of the DNA solution used in the electroporating process. The DNA may be purified by any known method, however, a preferred purification method is the use of DNA affinity purification. The purification of DNA, e.g., recombinant linear or plasmid DNA, using DNA binding resins and affinity reagents is well known and any of the known methods can be used in this invention (Vogelstein, B. and Gillespie, D., 1979, Proc. Natl. Acad. Sci. USA, 76:615; Callen, W., 1993, Strategies, 6:52-53). Commercially available DNA isolation and purification kits are also available from several sources including Stratagene (CLEARCUT Miniprep Kit), and Life Technologies (GLASSMAX DNA Isolation Systems). Suitable non-limiting methods of DNA purification include column chromatography (U.S. Pat. No. 5,707,812), the use of hydroxylated silica polymers (U.S. Pat. No. 5,693,785), rehydrated silica gel (U.S. Pat. No. 4,923,978), boronated silicates (U.S. Pat. No. 5,674,997), modified glass fiber membranes (U.S. Pat. Nos. 5,650,506; 5,438,127), fluorinated adsorbents (U.S. Pat. No. 5,625,054; U.S. Pat. No. 5,438,129), diatomaceous earth (U.S. Pat. No. 5,075,430), dialysis (U.S. Pat. No. 4,921,952), gel polymers (U.S. Pat. No. 5,106,966) and the use of chaotropic compounds with DNA binding reagents (U.S. Pat. No. 5,234,809). After purification, the DNA is eluted or otherwise resuspended in water, preferably distilled or deionized water, for use in electroporation at the concentrations of the invention. The use of low salt buffer solutions is also contemplated where the solution has low electrical conductivity, i.e., is compatible with the use of the high DNA concentrations of the invention with time constants greater than about 3.0 ms.

Any cells which can be transformed by electroporation may be used as host cells. Suitable host cells which can be transformed with heterologous DNA in the method of the invention include animal cells (Neumann et al., EMBO J., (1982), 1:841; Wong and Neumann, Biochem. Biophys. Res. Commun., (1982), 107:584; Potter et al., Proc. Natl. Acad. Sci., USA, (1984) 81:7161; Sugden et al., Mol. Cell. Biol., (1985), 5:410; Toneguzzo et al., Mol. Cell. Biol., (1986), 6:703; Pur-Kaspa et al., Mol. Cell. Biol., (1986), 6:716), plant cells (Fromm et al., Proc. Natl. Acad. Sci., USA, (1985), 82:5824; Fromm et al., Nature, (1986), 319:791; Ecker and Davis, Proc. Natl. Acad. Sci., USA, (1986) 83:5372) and bacterial cells (Chu et al., Nucleic Acids Res., (1987), 15:1311; Knutson and Yee, Anal. Biochem., (1987), 164:44). Prokaryotes are the preferred host cells for this invention. See also Andreason and Evans, Biotechniques, (1988), 6:650 which describes parameters which effect transfection efficiencies for varying cell lines. Suitable bacterial cells include E. coli (Dower et al., above; Taketo, Biochim. Biophys. Acta, (1988), 149:318), L. casei (Chassy and Flickinger, FEMS Microbiol. Lett., (1987), 44:173), Strept. lactis (Powell et al., Appl. Environ. Microbiol., (1988), 54:655; Harlander, Streptococcal Genetics, ed. J. Ferretti and R. Curtiss, III), page 229, American Society for Microbiology, Washington, D.C., (1987)), Strept. thermophilus (Somkuti and Steinberg, Proc. 4th Eur. Cong. Biotechnology, 1987, 1:412); Campylobacter jejuni (Miller et al., Proc. Natl. Acad. Sci., USA, (1988) 85:856), and other bacterial strains (Fielder and Wirth, Anal. Biochem., (1988), 170:38) including bacilli such as Bacillus subtilis, other enterobacteriaceae such as Salmonella typhimurium or Serratia marcesans, and various Pseudomonas species which may all be used as hosts. Suitable E. coli strains include JM101, E. coli K12 strain 294 (ATCC number 31,446), E. coli strain W3110 (ATCC number 27,325), E. coli X1776 (ATCC number 31,537), E. coli XL-1Blue (Stratagene), and E. coli B; however many other strains of E. coli, such as XL1-Blue MRF′, SURE, ABLE C, ABLE K, WM1100, MC1061, HB101, CJ136, MV1190, JS4, JS5, NM522, NM538, NM539, TG1 and many other species and genera of prokaryotes may be used as well.

Cells are made competent using known procedures. Sambrook et al., above, 1.76-1.81, 16.30.

The heterologous DNA is preferably in the form of a replicable transcription or expression vector, such as a phage or phagemid which can be constructed with relative ease and readily amplified. These vectors generally contain a promoter, a signal sequence, phenotypic selection genes, origins of replication, and other necessary components which are known to those of ordinary skill in this art. Construction of suitable vectors containing these components as well as the gene encoding one or more desired cloned polypeptides are prepared using standard recombinant DNA procedures as described in Sambrook et al., above. Isolated DNA fragments to be combined to form the vector are cleaved, tailored, and ligated together in a specific order and orientation to generate the desired vector.

The gene encoding the desired polypeptide (i.e., a peptide or a polypeptide with a rigid secondary structure or a protein) can be obtained by methods known in the art (see generally, Sambrook et al.). If the sequence of the gene is known, the DNA encoding the gene may be chemically synthesized (Merrfield, J. Am. Chem. Soc., 85:2149 (1963)). If the sequence of the gene is not known, or if the gene has not previously been isolated, it may be cloned from a cDNA library (made from RNA obtained from a suitable tissue in which the desired gene is expressed) or from a suitable genomic DNA library. The gene is then isolated using an appropriate probe. For cDNA libraries, suitable probes include monoclonal or polyclonal antibodies (provided that the cDNA library is an expression library), oligonucleotides, and complementary or homologous cDNAs or fragments thereof. The probes that may be used to isolate the gene of interest from genomic DNA libraries include cDNAs or fragments thereof that encode the same or a similar gene, homologous genomic DNAs or DNA fragments, and oligonucleotides. Screening the cDNA or genomic library with the selected probe is conducted using standard procedures as described in chapters 10-12 of Sambrook et al., above.

An alternative means to isolating the gene encoding the protein of interest is to use polymerase chain reaction methodology (PCR) as described in section 14 of Sambrook et al., above. This method requires the use of oligonucleotides that will hybridize to the gene of interest; thus, at least some of the DNA sequence for this gene must be known in order to generate the oligonucleotides.

After the gene has been isolated, it may be inserted into a suitable vector as described above for amplification, as described generally in Sambrook et al.

The DNA is cleaved using the appropriate restriction enzyme or enzymes in a suitable buffer. In general, about 0.2-1 μg of plasmid or DNA fragments is used with about 1-2 units of the appropriate restriction enzyme in about 20 μl of buffer solution. Appropriate buffers, DNA concentrations, and incubation times and temperatures are specified by the manufacturers of the restriction enzymes. Generally, incubation times of about one or two hours at 37° C. are adequate, although several enzymes require higher temperatures. After incubation, the enzymes and other contaminants are removed by extraction of the digestion solution with a mixture of phenol and chloroform, and the DNA is recovered from the aqueous fraction by precipitation with ethanol or other DNA purification technique.

To ligate the DNA fragments together to form a functional vector, the ends of the DNA fragments must be compatible with each other. In some cases, the ends will be directly compatible after endonuclease digestion. However, it may be necessary to first convert the sticky ends commonly produced by endonuclease digestion to blunt ends to make them compatible for ligation. To blunt the ends, the DNA is treated in a suitable buffer for at least 15 minutes at 15° C. with 10 units of the Klenow fragment of DNA polymerase I (Klenow) in the presence of the four deoxynucleotide triphosphates. The DNA is then purified by phenol-chloroform extraction and ethanol precipitation or other DNA purification technique.

The cleaved DNA fragments may be size-separated and selected using DNA gel electrophoresis. The DNA may be electrophoresed through either an agarose or a polyacrylamide matrix. The selection of the matrix will depend on the size of the DNA fragments to be separated. After electrophoresis, the DNA is extracted from the matrix by electroelution, or, if low-melting agarose has been used as the matrix, by melting the agarose and extracting the DNA from it, as described in sections 6.30-6.33 of Sambrook et al., supra.

The DNA fragments that are to be ligated together (previously digested with the appropriate restriction enzymes such that the ends of each fragment to be ligated are compatible) are put in solution in about equimolar amounts. The solution will also contain ATP, ligase buffer and a ligase such as T4 DNA ligase at about 10 units per 0.5 μg of DNA. If the DNA fragment is to be ligated into a vector, the vector is at first linearized by cutting with the appropriate restriction endonuclease(s). The linearized vector is then treated with alkaline phosphatase or calf intestinal phosphatase. The phosphatasing prevents self-ligation of the vector during the ligation step.

After ligation, the vector with the foreign gene now inserted is purified as described above and transformed into a suitable host cell such as those described above by electroporation using known and commercially available electroporation instruments and the procedures outlined by the manufacturers and described generally in Dower et al., above. A single electroporation reaction typically yields greater than 1×10¹⁰ transformants. However, more than one (a plurality) electroporation may be conducted to increase the amount of DNA which is transformed into the host cells. Repeated electroporations are conducted as described in the art. See Vaughan et al., above. The number of additional electroporations may vary as desired from several (2, 3, 4, . . . 10) up to tens (10, 20, 30, . . . 100) and even hundreds (100, 200, 300, . . . 1000). Repeated electroporations may be desired to increase the size of a combinatorial library, e.g. an antibody library, transformed into the host cells. With a plurality of electroporations, it is possible to produce a library having at least 1.0×10¹², even 2.0×10¹², different members (clones, DNA vectors such as phage, phagemids, plasmids, etc., cells, etc.).

Electroporation may be carried out using methods known in the art and described, f(r example, in U.S. Pat. Nos. 4,910,140; 5,186,800; 4,849,355; 5,173,158; 5,098,843; 5,422,272; 5,232,856; 5,283,194; 5,128,257; 5,750,373; 4,956,288 or any other known batch or continuous electroporation process together with the improvements of the invention.

Typically, electrocompetent cells are mixed with a solution of DNA at the desired concentration at ice temperatures. An aliquot of the mixture is placed into a cuvette and placed in an electroporation instrument, e.g., GENE PULSER (Biorad) having a typical gap of 0.2 cm. Each cuvette is electroporated as described by the manufacturer. Typical settings are: voltage=2.5 kV, resistance=200 ohms, capacitance=25 mF. The cuvette is then immediately removed, SOC media (Maniatis) is added, and the sample is transferred to a 250 mL baffled flask. The contents of several cuvettes may be combined after electroporation. The culture is then shaken at 37° C. to culture the transformed cells.

The transformed cells are generally selected by growth on an antibiotic, commonly tetracycline (tet) or ampicillin (amp), to which they are rendered resistant due to the presence of tet and/or amp resistance genes in the vector.

After selection of the transformed cells, these cells are grown in culture and the vector DNA (phage or phagemid vector containing a fusion gene library) may then be isolated. Vector DNA can be isolated using methods known in the art. Two suitable methods are the small scale preparation of DNA and the large-scale preparation of DNA as described in sections 1.25-1.33 of Sambrook et al., supra. The isolated DNA can be purified by methods known in the art such as that described in section 1.40 of Sambrook et al., above and as described above. This purified DNA is then analyzed by restriction mapping and/or DNA sequencing. DNA sequencing is generally performed by either the method of Messing et al., Nucleic Acids Res., 9:309 (1981) or by the method of Maxam et al., Meth. Enzymol., 65:499 (1980).

In the invention, the gene encoding a polypeptide (gene 1) is fused to a second gene (gene 2) such that a fusion protein is generated during transcription. Gene 2 is typically a coat protein gene of a filamentous phage, preferably phage Ml 3 or a related phage, and gene 2 is preferably the coat protein III gene or the coat protein VIII gene, or a fragment thereof. See U.S. Pat. No. 5,750,373; WO 95/34683. Fusion of genes 1 and 2 may be accomplished by inserting gene 2 into a particular site on a plasmid that contains gene 1, or by inserting gene 1 into a particular site on a plasmid that contains gene 2 using the standard techniques described above.

Alternatively, gene 2 may be a molecular tag for identifying and/or capturing and purifying the transcribed fusion protein. For example, gene 2 may encode for Herpes simplex virus glycoprotein D (Paborsky et al., 1990, Protein Engineering, 3:547-553) which can be used to affinity purify the fusion protein through binding to an anti-gD antibody. Gene 2 may also code for a polyhistidine, e.g., (his)₆ (Sporeno et al., 1994, J. Biol. Chem., 269:10991-10995; Stuber et al, 1990, Immunol. Methods, 4:121-152, Waeber et al., 1993, FEBS Letters, 324:109-112), which can be used to identify and/or purify the fusion protein through binding to a metal ion (Ni) column (QIAEXPRESS Ni-NTA protein Purification System, Quiagen, Inc.). Other affinity tags known in the art may be used and encoded by gene 2.

Insertion of a gene into a phage or phagemid vector requires that the vector be cut at the precise location that the gene is to be inserted. Thus, there must be a restriction endonuclease site at this location (preferably a unique site such that the vector will only be cut at a single location during restriction endonuclease digestion). The vector is digested, phosphatased, and purified as described above. The gene is then inserted into this linearized vector by ligating the two DNAs together. Ligation can be accomplished if the ends of the vector are compatible with the ends of the gene to be inserted. If the restriction enzymes are used to cut the vector and isolate the gene to be inserted create blunt ends or compatible sticky ends, the DNAs can be ligated together directly using a ligase such as bacteriophage T4 DNA ligase and incubating the mixture at 16° C. for 1-4 hours in the presence of ATP and ligase buffer as described in section 1.68 of Sambrook et al., above. If the ends are not compatible, they must first be made blunt by using the Klenow fragment of DNA polymerase I or bacteriophage T4 DNA polymerase, both of which require the four deoxyribonucleotide triphosphates to fill-in overhanging single-stranded ends of the digested DNA. Alternatively, the ends may be blunted using a nuclease such as nuclease S1 or mung-bean nuclease, both of which function by cutting back the overhanging single strands of DNA. The DNA is then religated using a ligase as described above. In some cases, it may not be possible to blunt the ends of the gene to be inserted, as the reading frame of the coding region will be altered. To overcome this problem, oligonucleotide linkers may be used. The linkers serve as a bridge to connect the vector to the gene to be inserted. These linkers can be made synthetically as double stranded or single stranded DNA using standard methods. The linkers have one end that is compatible with the ends of the gene to be inserted; the linkers are first ligated to this gene using ligation methods described above. The other end of the linkers is designed to be compatible with the vector for ligation. In designing the linkers, care must be taken to not destroy the reading frame of the gene to be inserted or the reading frame of the gene contained on the vector. In some cases, it may be necessary to design the linkers such that they code for part of an amino acid, or such that they code for one or more amino acids.

Between gene 1 and gene 2, DNA encoding a termination codon may be inserted, such termination codons are UAG (amber), UAA (ocher) and UGA (opel). (Microbiology, Davis et al. Harper & Row, New York, 1980, pages 237, 245-47 and 274). The termination codon expressed in a wild type host cell results in the synthesis of the gene 1 protein product without the gene 2 protein attached. However, growth in a suppressor host cell results in the synthesis of detectable quantities of fused protein. Such suppressor host cells contain a tRNA modified to insert an amino acid in the termination codon position of the mRNA thereby resulting in production of detectable amounts of the fusion protein. Such suppressor host cells are well known and described, such as E. coli suppressor strain (Bullock et al., BioTechniques 5:376-379 [1987]). Any acceptable method may be used to place such a termination codon into the mRNA encoding the fusion polypeptide.

The suppressible codon may be inserted between the first gene encoding a polypeptide, and a second gene encoding at least a portion of a phage coat protein. Alternatively, the suppressible termination codon may be inserted adjacent to the fusion site by replacing the last amino acid triplet in the polypeptide or the first amino acid in the phage coat protein. When the plasmid containing the suppressible codon is grown in a suppressor host cell, it results in the detectable production of a fusion polypeptide containing the polypeptide and the coat protein. When the plasmid is grown in a non-suppressor host cell, the polypeptide is synthesized substantially without fusion to the phage coat protein due to termination at the inserted suppressible triplet encoding UAG, UAA, or UGA. In the non-suppressor cell the polypeptide is synthesized and secreted from the host cell due to the absence of the fused phage coat protein which otherwise anchored it to the host cell.

Gene 1 may encode any polypeptide which can be expressed and displayed on the surface of a bacteriophage. The polypeptide is preferably a mammalian protein and may be, for example, selected from human growth hormone (hGH), N-methionyl human growth hormone, bovine growth hormone, parathyroid hormone, thyroxine, insulin A-chain, insulin B-chain, proinsulin, relaxin A-chain, relaxin B-chain, prorelaxin, glycoprotein hormones such as follicle stimulating hormone (FSH), thyroid stimulating hormone (TSH), leutinizing hormone (LH), glycoprotein hormone receptors, calcitonin, glucagon, factor VIII, an antibody, lung surfactant, urokinase, streptokinase, human tissue-type plasminogen activator (t-PA), bombesin, coagulation cascade factors including factor VII, factor IX, and factor X, thrombin, hemopoietic growth factor, tumor necrosis factor-alpha and -beta, enkephalinase, human serum albumin, mullerian-inhibiting substance, mouse gonadotropin-associated peptide, a microbial protein, such as betalactamase, tissue factor protein, inhibin, activin, vascular endothelial growth factor (VEGF), receptors for hormones or growth factors; integrin, thrombopoietin (TPO), protein A or D, rheumatoid factors, nerve growth factors such as NGF-alpha, platelet-growth factor, transforming growth factors (TGF) such as TGF-alpha and TGF-beta, insulin-like growth factor-I and —II, insulin-like growth factor binding proteins, CD-4, DNase, latency associated peptide, erythropoietin (EPO), osteoinductive factors, interferons such as interferon-alpha, -beta, and -gamma, colony stimulating factors (CSFs) such as M-CSF, GM-CSF, and G-CSF, interleukins (ILs) such as IL-1, IL-2, IL-31, IL-4, IL-6, L-8, IL-10, IL-12, superoxide dismutase; decay accelerating factor, viral antigen, HIV envelope proteins such as GP120, GP140, atrial natriuretic peptides A, B, or C, immunoglobulins, prostate specific antigen (PSA), prostate stem cell antigen (PSCA), as well as variants and fragments of any of the above-listed proteins. Other examples include Epidermal Growth Factor (EGF), EGF receptor, and peptides binding these and other proteins.

The first gene may encode a peptide containing as few as about 50-80 residues. These smaller peptides are useful in determining the antigenic properties of the peptides, in mapping the antigenic sites of proteins, etc. The first gene may also encode polypeptide having many hundreds, for example, 100, 200, 300, 400, and more amino acids. The first gene may also encode a polypeptide of one or more subunits containing more than about 100 amino acid residues which may be folded to form a plurality of rigid secondary structures displaying a plurality of amino acids capable of interacting with the target.

Known methods of phage and phagemid display of proteins, peptides and mutated variants thereof, including constructing a family of variant replicable vectors containing control sequences operably linked to a gene fusion encoding a fusion polypeptide, transforming suitable host cells, culturing the transformed cells to form phage particles which display the fusion polypeptide on the surface of the phage particle, contacting the recombinant phage particles with a target molecule so that at least a portion of the particle bind to the target, separating the particles which bind from those that do not, may be used in the method of the invention. See U.S. Pat. No. 5,750,373; WO 97/09446; U.S. Pat. Nos. 5,514,548; 5,498,538; 5,516,637; 5,432,018; WO 96/22393; U.S. Pat. Nos. 5,658,727; 5,627,024; WO 97/29185; O'Boyle et al, 1997, Virology, 236:338-347; Soumillion et al, 1994, Appl. Biochem. Biotech., 47:175-190; O'Neil and Hoess, 1995, Curr. Opin. Struct. Biol., 5:443-449; Makowski, 1993, Gene, 128:5-11; Dunn, 1996, Curr. Opin. Struct. Biol., 7:547-553; Choo and Klug, 1995, Curr. Opin. Struct. Biol., 6:431-436; Bradbury and Cattaneo, 1995, TINS, 18:242-249; Cortese et al., 1995, Curr. Opin. Struct. Biol., 6:73-80; Allen et al., 1995, TIBS, 20:509-516; Lindquist and Naderi, 1995, FEMS Micro. Rev., 17:33-39; Clarkson and Wells, 1994, Tibtech, 12:173-184; Barbas, 1993, Curr. Opin. Biol., 4:526-530; McGregor, 1996, Mol. Biotech., 6:155-162; Cortese et al., 1996, Curr. Opin. Biol., 7:616-621; McLafferty et al., 1993, Gene, 128:29-36. The phage/phagemid display of the variants may be on the N-terminus or on the C-terminus of a phage coat protein or portion thereof. Further, the phage/phagemid display may use natural or mutated coat proteins, for example non-naturally occurring variants of a filamentous phage coat protein III or VIII, or a de novo designed coat protein. See for example, WO00/06717 published Feb. 10, 2000, which is expressly incorporated herein by reference.

In one embodiment, gene 1 encodes the light chain or the heavy chain of an antibody or fragments thereof, such Fab, F(ab′)₂, Fv, diabodies, linear antibodies, etc. Gene 1 may also encode a single chain antibody (scFv). The preparation of libraries of antibodies or fragments thereof is well known in the art and any of the known methods may be used to construct a family of transformation vectors which may be transformed into host cells using the method of the invention Libraries of antibody light and heavy chains in phage (Huse et al, 1989, Science, 246:1275) and as fusion proteins in phage or phagemid are well known and can be prepared according to known procedures. See Vaughan et al., Barbas et al., Marks et al., Hoogenboom et al., Griffiths et al., de Kruif et al., noted above, and WO 98/05344; WO 98/15833; WO 97/47314; WO 97/44491; WO 97/35196; WO 95/34648; U.S. Pat. Nos. 5,712,089; 5,702,892; 5,427,908; 5,403,484; 5,432,018; 5,270,170; WO 92/06176; U.S. Pat. No. 5,702,892. Reviews have also published. Hoogenboom, 1997, Tibtech, 15:62-70; Neri et al., 1995, Cell Biophysics, 27:47; Winter et al., 1994, Annu. Rev. Immunol., 12:433-455; Soderlind et al., 1992, Immunol. Rev., 130:109-124; Jefferies, 1998, Parasitology, 14:202-206.

Specific antibodies contemplated as being encoded by gene 1 include antibodies and antigen binding fragments thereof which bind to human leukocyte surface markers, cytokines and cytokine receptors, enzymes, etc. Specific leukocyte surface markers include CD1a-c, CD2, CD2R, CD3-CD10, CD11a-c, CDw12, CD13, CD14, CD15, CD15s, CD16, CD16b, CDw17, CD18-C41, CD42a-d, CD43, CD44, CD44R, CD45, CD45A, CD45B, CD45O, CD46-CD48, CD49a-f, CD50-CD51, CD52, CD53-CD59, CDw60, CD61, CD62E, CD62L, CD62P, CD63, CD64, CDw65, CD66a-e, CD68-CD74, CDw75, CDw76, CD77, CDw78, CD79a-b, CD80-CD83, CDw84, CD85-CD89, CDw90, CD91, CDw92, CD93-CD98, CD99, CD99R, CD100, CDw101, CD102-CD106, CD107a-b, CDw108, CDw109, CD115, CDw116, CD117, CD119, CD120a-b, CD121a-b, CD122, CDw124, CD126-CD129, and CD130. Other antibody binding targets include cytokines and cytokine superfamily receptors, hematopoietic growth factor superfamily receptors and preferably the extracellular domains thereof, which are a group of closely related glycoprotein cell surface receptors that share considerable homology including frequently a WSXWS domain and are generally classified as members of the cytokine receptor superfamily (see e.g. Nicola et al., Cell, 67:1-4 (1991) and Skoda, R. C. et al. EMBO J. 12:2645-2653 (1993)). Generally, these targets are receptors for interleukins (L) or colony-stimulating factors (CSF). Members of the superfamily include, but are not limited to, receptors for: IL-2 (b and g chains) (Hatakeyama et al., Science, 244:551-556 (1989); Takeshita et al., Science, 257:379-382 (1991)), IL-3 (Itoh et al., Science, 247:324-328 (1990); Gorman et al., Proc. Natl. Acad. Sci. USA, 87:5459-5463 (1990); Kitamura et al., Cell, 66:1165-1174 (1991a); Kitamura et al., Proc. Natl. Acad. Sci. USA, 88:5082-5086 (1991b)), IL-4 (Mosley et al., Cell, 59:335-348 (1989), IL-5 (Takaki et al., EMBO J., 9:4367-4374 (1990); Tavernier et al., Cell, 66:1175-1184 (1991)), IL-6 (Yamasaki et al., Science, 241:825-828 (1988); Hibi et al., Cell, 63:1149-1157 (1990)), IL-7 (Goodwin et al., Cell, 60:941-951 (1990)), IL-9 (Renault et al., Proc. Natl. Acad. Sci. USA, 89:5690-5694 (1992)), granulocyte-macrophage colony-stimulating factor (GM-CSF) (Gearing et al., EMBO J., 8:3667-3676 (1991); Hayashida et al., Proc. Natl. Acad. Sci. USA, 244:9655-9659 (1990)), granulocyte colony-stimulating factor (G-CSF) (Fukunaga et al., Cell, 61:341-350 (1990a); Fukunaga et al., Proc. Natl. Acad. Sci. USA, 87:8702-8706 (1990b); Larsen et al., J. Exp. Med., 172:1559-1570 (1990)), EPO (D'Andrea et al., Cell, 57:277-285 (1989); Jones et al., Blood, 76:31-35 (1990)), Leukemia inhibitory factor (LIF) (Gearing et al., EMBO J., 10:2839-2848 (1991)), oncostatin M (OSM) (Rose et al., Proc. Natl. Acad. Sci. USA, 88:8641-8645 (1991)) and also receptors for prolacti (Boutin et al., Proc. Natl. Acad. Sci. USA, 88:7744-7748 (1988); Edery et al., Proc. Natl. Acad. Sci. USA, 86:2112-2116 (1989)), growth hormone (GH) (Leung et al., Nature, 330:537-543 (1987)), ciliary neurotrophic factor (CNTF) (Davis et al., Science, 253:59-63 (1991) and c-Mpl (M. Souyri et al., Cell 63:1137 (1990); I. Vigon et al., Proc. Natl. Acad. Sci. 89:5640 (1992)). Still other targets for antibodies made by the invention are erb2, erb3, erb4, IL-10, IL-12, IL-13, IL-15, etc. Any of these antibodies, antibody fragments, cytokines, receptors, enzymes, cell surface marker proteins, etc. may be encoded by the first gene.

A library of fusion genes encoding the desired fusion protein library may be produced by a variety of methods known in the art. These methods include but are not limited to oligonucleotide-mediated mutagenesis and cassette mutagenesis. The method of the invention uses a limited codon set to prepare the libraries of the invention. The limited codon set allows for a wild-type amino acid and a scanning amino acid at each of the predetermined positions of the polypeptide. For example, if the scanning amino acid is alanine, the limited codon set would code for a wild-type amino acid and alanine as possible amino acids at each of the predetermined positions. Tables 1-6, below, provide examples of how to prepare the limited codon sets which are used in this invention. The DNA degeneracies are represented by IUB code (K=G/T, M=A/C, N=A/C/G/T, R=A(G, IS=G/C, W=A/T, Y═C/T). Tables of DNA degeneracies for limited codon sets for the use of other scanning amino acids can be readily constructed from the known degeneracies of the genetic code following the guidance of these examples and the general disclosure herein.

TABLE 1
Shotgun Ala Scanning Codons

| wt* aa | shotgun codon | shotgun aa's |
|---|---|---|
| A | GST | A/G |
| C | KST | A/C/G/S |
| D | GMT | A/D |
| E | GMA | A/E |
| F | KYT | A/F/S/V |
| G | GST | A/G |
| H | SMT | A/G/D/P |
| I | RYT | A/I/T/V |
| K | RMA | A/K/E/T |
| L | SYT | A/L/P/V |
| M | RYG | A/M/T/V |
| N | RMC | A/N/D/T |
| P | SCA | A/P |
| Q | SMA | A/Q/E P |
| R | SST | A/R/G/P |
| S | KCC | A/S |
| T | RCT | A/T |
| V | GYT | A/V |
| W | KSG | A/W/G/S |
| Y | KMT | A/Y/D/S |

TABLE 2
Shotgun Arg Scanning codons

| wt* aa | shotgun codon | shotgun aa's |
|---|---|---|
| A | SSC | R/A/P/G |
| C | YGT | R/C |
| D | SRC | R/D/H/G |
| E | SRA | R/E/G/Q |
| F | YKC | R/F/L/C |
| G | SGT | R/G |
| H | CRT | R/H |
| I | AKA | R/I |
| K | ARA | R/K |
| L | CKC | R/L |
| M | AKG | R/M |
| N | MRC | R/N/H/S |
| P | CSA | R/P |
| Q | CRA | R/Q |
| R* | CGT | R |
| S | AGM | R/S |
| T | ASG | R/T |
| V | SKT | R/V/G/L |
| W | YGG | R/W |
| Y | YRT | R/Y/C/H |

TABLE 3
Shotgun Glu Scanning Codons

| wt* aa | shotgun codon | shotgun aa's |
|---|---|---|
| A | GMA | E/A |
| C | YRK | E/C/W/Y/R/H/Q/Amber stop |
| D | GAM | E/D |
| E* | GAA | E |
| F | KWS | E/F/Y/L/D/V/Amber stop |
| G | GRG | E/G |
| H | SAM | E/H/Q |
| I | RWA | E/I/V/K |
| K | RAA | E/K |
| L | SWG | E/L/V/Q |
| M | RWG | E/M/K/V |
| N | RAM | E/N/K/D |
| P | SMA | E/P/Q/A |
| Q | SAA | E/Q |
| R | SRA | E/R/G/Q |
| S | KMG | E/S/A/Amber stop |
| T | RMG | E/T/K/A |
| V | GWA | E/V |
| W | KRG | E/W/G/Amber stop |
| Y | KAS | E/Y/D/Amber stop |

TABLE 4
Shotgun Leu Scanning Codons

| wt* aa | shotgun codon | shotgun aa's |
|---|---|---|
| A | SYG | L/A/V/P |
| C | YKT | L/C/F/R |
| D | SWC | L/D/H/V |
| E | SWG | L/E/V/Q |
| F | YTC | L/F |
| G | SKG | L/G/V/R |
| H | CWT | L/H |
| I | MTC | L/I |
| K | MWG | L/K/M/Q |
| L* | CTG | L |
| M | MTG | L/M |
| N | MWG | L/N/H/I |
| P | CYG | L/P |
| Q | CWA | L/Q |
| R | CKC | L/R |
| S | TYG | L/S |
| T | MYC | L/T/I/P |
| V | STG | L/V |
| W | TKG | L/W |
| Y | TWS | L/Y/F/Amber stop |

TABLE 5
Shotgun Phe Scanning Codons

| wt* aa | shotgun codon | shotgun aa's |
|---|---|---|
| A | KYC | F/A/V/S |
| C | TKC | F/C |
| D | KWC | F/D/Y/V |
| E | KWM | F/E/V/Y |
| F* | TTC | F |
| G | KKC | F/G/V/C |
| H | YWC | F/H/L/Y |
| I | WTC | F/I |
| K | WWS | F/K/I/M/Y/Amber stop |
| L | YTC | F/L |
| M | WTS | F/M/I/L |
| N | WWC | F/N/Y/I |
| P | YYC | F/P/L/S |
| Q | YWS | F/Q/L/Y/Amber stop |
| R | YKC | F/R/C/L |
| S | TYC | F/S |
| T | WYC | F/T/I/S |
| V | KTC | F/V |
| W | TKS | F/W/C/L |
| Y | TWC | F/Y |

TABLE 6
Shotgun Ser Scanning Codons

| | | |
|---|---|---|
| A | KCC | S/A |
| C | RGC | S/C |
| D | KMC | S/D/A/Y |
| E | KMG | S/B/A/Amber stop |
| F | TYC | S/F |
| G | RGT | S/G |
| H | MRC | S/H/R/N |
| I | AKC | S/I |
| K | ARM | S/K/R/N |
| L | TYG | S/L |
| M | AKS | S/M/R/I |
| N | ARC | S/N |
| P | YCT | S/P |
| Q | YMG | S/Q/P/Amber stop |
| R | MGT | S/R |
| S* | TCC | S |
| T | WCG | S/T |
| V | KYT | S/V/F/A |
| W | TSG | S/W |
| Y | TMC | S/Y |

*wt = wild-type

In one embodiment, the limited codon set allows for only the scanning residue and a wild-type residue at each of the predetermined polypeptide positions. Such limited codon sets may be produced using oligonucleotides prepared from trinucleotide synthon units using methods known in the art. See for example, Gayan et al., Chem. Biol., 5: 519-527. Use of trinucleotides removes the wobble in the codons which codes for additional amino acid residues. This embodiment enables a wild-type to scanning residue ratio of 1:1 at each scanned position.

Surprisingly, the use of a codon set allowing two or more, e.g., four, amino acid residues and possibly a stop codon, does not affect the resulting analysis of wild-type versus scanning residue frequency or the ability of the method of the invention to identify polypeptide positions which are structurally and/or functionally important. The results obtained by the present invention are particularly surprising in view of arguments that ΔΔG_{mut-wt} values derived from single alanine mutants are a poor measure of individual side chain binding contributions, because cooperative intramolecular interactions likely make most large binding interfaces extremely non-additive (Greenspan and Di Cera, 1999, Nature Biotechnology 17:936). The invention allows construction and analysis of every possible multiple scanning amino acid, e.g., alanine, mutant covering a large portion of a structural binding epitope, in a combinatorial manner. Even in this extremely diverse background, the functional contributions of individual side chains were remarkably similar to their contributions in the fixed wild-type, e.g., hGH, background (See Example 1). While non-additive effects should certainly be considered, the major contributors of binding energy at a protein-ligand, e.g. the hGH-hGHbp, interface act independently in an essentially additive manner. The results obtained for this invention are in good agreement with previous studies that have demonstrated additivity in hGH site-I (Lowman and Wells, 1993, J. Mol. Biol. 234:564) and many other proteins (Wells, 1990, Biochemistry 29:8509).

Oligonucleotide-mediated mutagenesis is a preferred method for preparing a library of fusion genes. This technique is well known in the art as described by Zoller et al., Nucleic Acids Res., 10: 6487-6504 (1987). Briefly, gene 1 is altered by hybridizing an oligonucleotide encoding the desired mutation to a DNA template, where the template is the single-stranded form of the plasmid containing the unaltered or native DNA sequence of gene 1. After hybridization, a DNA polymerase, used to synthesize an entire second complementary strand of the template, will thus incorporate the oligonucleotide primer, and will code for the selected alteration in gene 1.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al., Proc. Natl. Acad. Sci. USA, 75: 5765 (1978).

The DNA template is preferably generated by those vectors that are either derived from bacteriophage M13 vectors (the commercially available M13 mp18 and M13 mp19 vectors are suitable), or those vectors that contain a single-stranded phage origin of replication as described by Viera et al., Meth. Enzymol., 153: 3 (1987). Thus, the DNA that is to be mutated can be inserted into one of these vectors in order to generate single-stranded template. Production of the single-stranded template is described in sections 4.21-4.41 of Sambrook et al., above.

To alter the native DNA sequence, the oligonucleotide is hybridized to the single stranded template under suitable hybridization conditions. A DNA polymerizing enzyme, usually T7 DNA polymerase or the Kienow fragment of DNA polymerase I, is then added to synthesize the complementary strand of the template using the oligonucleotide as a primer for synthesis. A heteroduplex molecule is thus formed such that one strand of DNA encodes the mutated form of gene 1, and the other strand (the original template) encodes the native, unaltered sequence of gene 1. This heteroduplex molecule is then transformed into a suitable host cell, usually a prokaryote such as E. coli JM101. After growing the cells, they are plated onto agarose plates and screened using the oligonucleotide primer radiolabelled with 32-phosphate to identify the bacterial colonies that contain the mutated DNA.

The method described immediately above may be modified such that a homoduplex molecule is created wherein both strands of the vector contain the mutation(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thiodeoxyribocytosine called dCTP-(aS) (which can be obtained from Amersham). This mixture is added to the template-oligonucleotide complex. Upon addition of DNA polymerase to this mixture, a strand of DNA identical to the template except for the mutated bases is generated. In addition, this new strand of DNA will contain dCTP-(aS) instead of dCTP, which serves to protect it from restriction endonuclease digestion. After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. coli JM101, as described above.

Mutants with more than one amino acid to be substituted may be generated in one of several ways. If the amino acids are located close together in the polypeptide chain, they may be mutated simultaneously using one oligonucleotide that codes for all of the desired amino acid substitutions. If, however, the amino acids are located some distance from each other (separated by more than about ten amino acids), it is more difficult to generate a single oligonucleotide that encodes all of the desired changes. Instead, one of two alternative methods may be employed.

In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant. The first round is as described for the single mutants: wild-type DNA is used for the template, an oligonucleotide encoding the first desired amino acid substitution(s) is annealed to this template, and the heteroduplex DNA molecule is then generated. The second round of mutagenesis utilizes the mutated DNA produced in the first round of mutagenesis as the template. Thus, this template already contains one or more mutations. The oligonucleotide encoding the additional desired amino acid substitution(s) is then annealed to this template, and the resulting strand of DNA now encodes mutations from both the first and second rounds of mutagenesis. This resultant DNA can be used as a template in a third round of mutagenesis, and so on.

Cassette mutagenesis is also a preferred method for preparing a library of fusion genes. The method is based on that described by Wells et al., Gene, 34:315 (1985). The starting material is the vector comprising gene 1, the gene to be mutated. The codon(s) in gene 1 to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in gene 1. After the restriction sites have been introduced into the vector, the vector is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are compatible with the ends of the linearized vector, such that it can be directly ligated to the vector. This vector now contains the mutated DNA sequence of gene 1.

In a preferred embodiment, gene 1 is linked to gene 2 encoding at least a portion of a phage coat protein. Preferred coat protein genes are the genes encoding coat protein III and coat protein VIII of filamentous phage specific for E. coli, such as M13, fl and fd phage. Transfection of host cells with a replicable expression vector library which encodes the gene fusion of gene 1 and gene 2 and production of a phage or phagemid particle library (or a fusion protein library) according to standard procedures provides phage or phagemid particles in which the variant polypeptides encoded by gene 1 are displayed on the surface of the virus particles.

Suitable phage and phagemid vectors for use in this invention include all known vectors for phage display. Additional examples include pComb8 (Gram, H., Marconi, L. A., Barbas, C. F., Collet, T. A., Lerner, R. A., and Kang, A. S. (1992) Proc. Natl. Acad. Sci. USA 89:3576-3580); pC89 (Felici, F., Catagnoli, L., Musacchio, A., Jappelli, R., and Cesareni, G. (1991) J. Mol. Biol. 222:310-310); pIF4 (Bianchi, E., Folgori, A., Wallace, A., Nicotra, M., Acali, S., Phalipon, A., Barbato, G., Bazzo, R., Cortese, R., Felici, F., and Pessi, A. (1995) J. Mol. Biol. 247:154-160); PM48, PM52, and PM54 (Iannolo, G., Minenkova, O., Petruzzelli, R., and Cesareni, G. (1995) J. Mol. Biol, 248:835-844); fdH (Greenwood, J., Willis, A. E., and Perham, R. N. (1991) J. Mol. Biol,. 220:821-827); pfd8SHU, pfd8SU, pfd8SY, and fdISPLAY8 (Malik, P. and Perham, R. N. (1996) Gene, 171:49-51); “88” (Smith, G. P. (1993) Gene, 128:1-2); f88.4 (Zhong, G., Smith, G. P., Berry, J. and Brunham, R. C. (1994) J. Biol. Chem, 269:24183-24188); p8V5 (Affymax); MB1, MB20, MB26, MB27, MB28, MB42, MB48, MB49, MB56: Markland, W., Roberts, B. L., Saxena, M. J., Guterman, S. K., and Ladner, R. C. (1991) Gene, 109:13-19). Similarly, any known helper phage may be used when a phagemid vector is employed in the phage display system. Examples of suitable helper phage include M13-KO7 (Pharmacia), M13-VCS (Stratagene), and R408 (Stratagene).

Transfection is preferably by electroporation. Preferably, viable cells are concentrated to about 1×10¹¹ to about 4×10¹¹ cfu/mL. Preferred cells which may be concentrated to this range are the SS320 cells described below. In this embodiment, cells are grown in culture in standard culture broth, optionally for about 6-48 hrs (or to OD₆₀₀=0.6-0.8) at about 37° C., and then the broth is centrifuged and the supernatant removed (e.g. decanted). Initial purification is preferably by resuspending the cell pellet in a buffer solution (e.g. HEPES pH 7.4) followed by recentrifugation and removal of supernatant. The resulting cell pellet is resuspended in dilute glycerol (e.g. 5-20% v/v) and again recentrifuged to form a cell pellet and the supernatant removed. The final cell concentration is obtained by resuspending the cell pellet in water or dilute glycerol to the desired concentration. These washing steps have an effect on cell survival, that is on the number of viable cells in the concentrated cell solution used for electroporation. It is preferred to use cells which survive the washing and centrifugation steps in a high survival ratio relative to the number of starting cells prior to washing. Most preferably, the ratio of the number of viable cells after washing to the number of viable cells prior to washing is 1.0, i.e., there is no cell death. However, the survival ratio may be about 0.8 or greater, preferably about 0.9-1.0.

A particularly preferred recipient cell is the electroporation competent E. coli strain of the present invention, which is E. coli strain MC 1061 containing a phage F′ episome. Any F′ episome which enables phage replication in the strain may be used in the invention. Suitable episomes are available from strains deposited with ATCC or are commercially available (CJ236, CSH18, DH5alphaF′, JM101, JM103, JM105, JM107, JM109, JM110), KS1000, XL1-BLUE, 71-18 and others). Strain SS320 was prepared by mating MC1061 cells with XL1-BLUE cells under conditions sufficient to transfer the fertility episome (F′ plasmid) of XL1-BLUE into the MC1061 cells. In general, mixing cultures of the two cell types and growing the mixture in culture medium for about one hour at 37° C. is sufficient to allow mating and episome transfer to occur. The new resulting E. coli strain has the genotype of MC1061 which carries a streptomycin resistance chromosomal marker and the genotype of the F′ plasmid which confers tetracycline resistance. The progeny of this mating is resistant to both antibiotics and can be selectively grown in the presence of streptomycin and tetracycline. Strain SS320 has been deposited with the American Type Culture Collection (ATCC), 10801 University Boulevard, Manassas, Va., USA on Jun. 18, 1998 and assigned Deposit Accession No. 98795.

SS320 cells have properties which are particularly favorable for electroporation. SS320 cells are particularly robust and are able to survive multiple washing steps with higher cell viability than most other electroporation competent cells. Other strains suitable for use with the higher cell concentrations include TB1, MC1061, etc. These higher cell concentrations provide greater transformation efficiency for the process of the invention.

The use of higher DNA concentrations during electroporation (about 10×) increases the transformation efficiency and increases the amount of DNA transformed into the host cells. The use of higher cell concentrations also increases the efficiency (about 10×). The larger amount of transferred DNA produces larger libraries having greater diversity and representing a greater number of unique members of a combinatorial library.

The construction of libraries, for example a library of fusion genes encoding fusion polypeptides, necessarily involves the introduction of DNA fragments representing the library into a suitable vector to provide a family or library of vectors. In the case of cassette mutagenesis, the synthetic DNA is a double stranded cassette while in fill-in mutagenesis the synthetic DNA is single stranded DNA. In either case, the synthetic DNA is incorporated into a vector to yield a reaction product containing closed circular double stranded DNA which can be transformed into a cell to produce a library.

The transformed cells are generally selected by growth on an antibiotic, commonly tetracycline (tet) or ampicillin (amp), to which they are rendered resistant due to the presence of tet and/or amp resistance genes in the vector.

The transformed cells, these cells are grown in culture and the vector DNA may then be isolated. Phage or phagemid vector DNA can be isolated using methods known in the art, for example, as described in Sambrook et al., Molecular Cloning: A Laboratory Manual, 2nd edition, (1989) Cold Spring Harbor Laboratory Press, Cold Spring Harbor, N.Y.

The isolated DNA can be purified by methods known in the art such as that described in section 1.40 of Sambrook et al., above and as described above. This purified DNA can then be analyzed by DNA sequencing. DNA sequencing may be performed by the method of Messing et al., Nucleic Acids Res., 9:309 (1981), the method of Maxam et al., Meth. Enzymol., 65:499 (1980), or by any other known method.

The invention also contemplates producing product polypeptides which have been obtained by culturing a host cell transformed with a replicable expression vector, where the replicable expression vector contains DNA encoding a product polypeptide operably linked to a control sequence capable of effecting expression of the product polypeptide in the host cell; where the DNA encoding the product polypeptide has been obtained by:

(a) constructing a library of expression vectors containing fusion genes encoding a plurality of fusion proteins, wherein the fusion proteins comprise a polypeptide portion fused to at least a portion of a phage coat protein, the polypeptide portions of the fusion proteins differ at a predetermined number of amino acid positions, and the fusion genes encode at most four different amino acids at each predetermined amino acid position;

(b) transforming suitable host cells with the library of expression vectors;

(c) culturing the transformed host cells under conditions suitable for forming recombinant phage or phagemid particles displaying variant fusion proteins on the surface thereof;

(d) contacting the recombinant particles with a target molecule so that at least a portion of the particles bind to the target molecule;

(e) separating particles that bind to the target molecule from those that do not bind;

(f) selecting one of the variant as the product polypeptide and cloning DNA encoding the product polypeptide into the replicable expression vector; and recovering the expressed product polypeptide. Methods of construction of a replicable expression vector and the production and recovery of product polypeptides is generally known in the art.

U.S. Pat. No. 5,750,373 describes generally how to produce and recover a product polypeptide by culturing a host cell transformed with a replicable expression vector (e.g., a phagemid) where the DNA encoding the polypeptide has been obtained by steps (a)-(f) above using conventional helper phage where a minor amount (<20%, preferably <10%, more preferably <1%) of the phage particles display the fusion protein on the surface of the particle. Any suitable helper phage may be used to produce recombinant phagemid particles, e.g., VCS, etc. One of the variant polypeptides obtained by the phage display process may be selected for larger scale production by recombinant expression in a host cell. Culturing of a host cell transformed with a replicable expression vector which contains DNA encoding a product polypeptide which is the selected variant operably linked to a control sequence capable of effecting expression of the product polypeptide in the host cell and then recovering the product polypeptide using known methods is part of this invention.

### EXAMPLES

As a representative example of the generality and principles of shotgun scanning, the high affinity site (site-i) of human growth hormone (hGH) was mapped for binding to its receptor (hGHbp). Crystallographic data was used to identify 19 hGH side chains that become at least 60% buried upon binding to hGHbp and together comprise a substantial portion of the structural binding epitope (A. M. de Vos et al, 1992, Science 255:306). These side chains are located on three non-contiguous stretches of primary sequence, but together they form a contiguous patch in the three-dimensional structure. This library replaced buried residues with a “shotgun code” of degenerate codons (see Table 1). Ideally, a binomial mutagenesis strategy would allow only the wild-type amino acid or alanine at each varied position. Due to degeneracy in the genetic code, some residues also required two other amino acid substitutions. We applied a binomial analysis to all mutations, by considering levels of wild-type or alanine in each position.

Substituting amino acids with alanine eliminates all sidechain atoms past the beta-carbon. This loss can be evaluated with a binding measurement of the mutant protein to evaluate contribution of that sidechain on the structure and function of the protein (Clackson and Wells, 1995 Science 267:383). The perturbation wrought by each alanine substitution was evaluated here en masse, using equilibrium binding to receptor-coated plates as the library selection. The phage-displayed library was subjected to selections for binding to either an anti-hGH antibody or to the hGHbp extracellular domain. The antibody bound to a hGH epitope distant from site-1, and required correct hGH folding for binding. This antibody selected hGH structure, independently of the selection for protein function.

Several hundred binding clones were sequenced from each selection, and the occurrence of wild-type or alanine was tabulated for each mutated position. At positions that encoded additional side chains, the analysis focused entirely on the wild-type and alanine. However, shotgun scanning with amino acids other than alanine is also useful.

Culture supernatant containing phage particles was used as template for a PCR that amplified the hGH gene and incorporated M13(−21) and M13R universal sequencing primers. Phage from the library were cycled through rounds of binding selection with hGHbp or anti-hGH monoclonal antibody 3F6.B1.4B1 (Jin et al, 1992, J. Mol. Biol. 226:851) coated on 96-well Maxisorp immunoplates (NUNC) as the capture target. Phage were propagated in E. coli XL1-blue with the addition of M13-VCS helper phage (Stratagene). After one (antibody sort) or three (hGHbp sort) rounds of selection, individual clones were grown in 500 μL cultures in a 96-well format. The culture supernatants were used directly in phage ELISAs to detect phage-displayed hGH variants that bound to either hGHbp or anti-hGH antibody 3F6.B 1.4B 1 immobilized on a 96-well Maxisorp immunoplate The amplified DNA fragment was used as the template in Big-Dye™ terminator sequencing reactions, which were analyzed on an ABI377 sequencer (PE-Biosystems). All reactions were performed in a 96-well format. The program “SGcount” aligned each DNA sequence against the wild-type DNA sequence using a Needleman-Wunch pairwise alignment algorithm, translated each aligned sequence of acceptable quality, and then tabulated the occurrence of each natural amino acid at each position. Additionally, “Sgcount” reported the presence of any sequences containing identical amino acids at all mutated positions (siblings). The antibody sort (175 total sequences) did not contain any siblings, while the hGHbp sort (330 total sequences,) contained 16 siblings representing 5 unique sequences.

The program “SGcount” was written in C and compiled and tested on Compaq/DEC alpha under Digital Unix 4.0D. The source is available (email: ckw@gene.com) and compiles without modification on most Unix systems. See also Weiss et al, 2000, PNAS 97:8950-8954 and W(D 0015666.

The wild-type frequency (F) was calculated as follows:

F=Σn_{wild-type}/Σ(n_{wild-type}+nₐₗₐₙᵢₙₑ)

For each side chain, we assumed that the difference between the wild-type frequency for the hGHbp selection (F_{bp}) and the antibody selection (F_{α}) is a measure of that side chain's contribution to the functional binding epitope. We used the F_{bp} and F_{α} values to calculate a “function parameter” (P_{f}) for each side chain. The P_{f} and associated standard error (SE) were calculated as follows:

$$For {F}_{bp} > {F}_{α} , {P}_{f} = ( {F}_{bp} - {F}_{α} ) / {( 1 - {F}_{α} ) \\ [ SE ( {P}_{f} ) ]}^{2} = \frac{{( 1 - {F}_{bp} )}^{2}}{{( 1 - {F}_{α} )}^{2}} [ \frac{{σ}_{bp}^{2}}{{( 1 - {F}_{bp} )}^{2}} + \frac{{σ}_{α}^{2}}{{( 1 - {F}_{α} )}^{2}} ]$$

$$For {F}_{bp} < {F}_{α} , {P}_{f} = ( {F}_{bp} - {F}_{α} ) / {{F}_{α} \\ [ SE ( {P}_{f} ) ]}^{2} = \frac{{F}_{bp}^{2}}{{F}_{α}^{2}} [ \frac{{σ}_{bp}^{2}}{{F}_{bp}^{2}} + \frac{{σ}_{α}^{2}}{{F}_{α}^{2}} ]$$

σ²_{bp} is the variance of F_{bp} and is approximated by F_{bp}(1−F_{bp})/n_{bp}.

σ²_{α} is the variance of F_{α} and is approximated by F_{α}(1−F_{α})/n_{α}.

If F_{bp}=F_{α}, the side chain does not contribute to the functional epitope and P_{f}=0.

If F_{bp}>F_{α}, the side chain contributes favorably to the functional epitope and P_{f}>0.

Positive P_{f} values are a normalized measure of where F_{bp} lies relative to F_{α} and one.

The maximum possible P_{f} value is P_{f}=1, which occurs when F_{bp}=1.

If F_{bp}<F_{α}, the side chain contributes unfavorably to the functional epitope and P_{f}<0.

Negative P_{f} values are a normalized measure of where F_{bp} lies relative to F_{α} and zero.

The minimum possible P_{f} value is P_{f}=−1, which occurs when F_{bp}=0.

For each selection, the sequence data was used to calculate the wild-type frequency at each position (B. Virnekas et al., 1994, Nucleic Acids Res. 22:5600; Gaytan et al., Chem. Biol. 5:519). The wild-type frequency compares the occurrence of a wild-type side chain relative to alanine, and thus, correlates with a given side chain's contribution to the selected trait (i.e. binding to antibody or hGHbp). The wild-type frequency for a large, favorable contribution to the binding interaction should approach 1.0 (100% enrichment for the wild-type side chain). The wild-type frequency for a large, negative contribution to binding should approach 0.0 (selection against the wild-type side chain). Because hGHbp contacts the mutated side chains, but the monoclonal antibody does not, the difference between the wild-type frequencies calculated from the two selections can be used to map the functional epitope of hGH for binding to hGHbp. While both selections are sensitive to bias in the naive library, expression biases and global structural perturbations, only the hGHbp selection is sensitive to the loss or gain of binding energy due to contacts with mutated residues in the structural epitope. We used the difference between the wild-type frequency from the antibody selection (F_{α}) and the hGHbp selection (F_{bp}) to calculate a “function parameter” (P_{f}) that normalizes each side chain's contribution to the functional binding epitope.

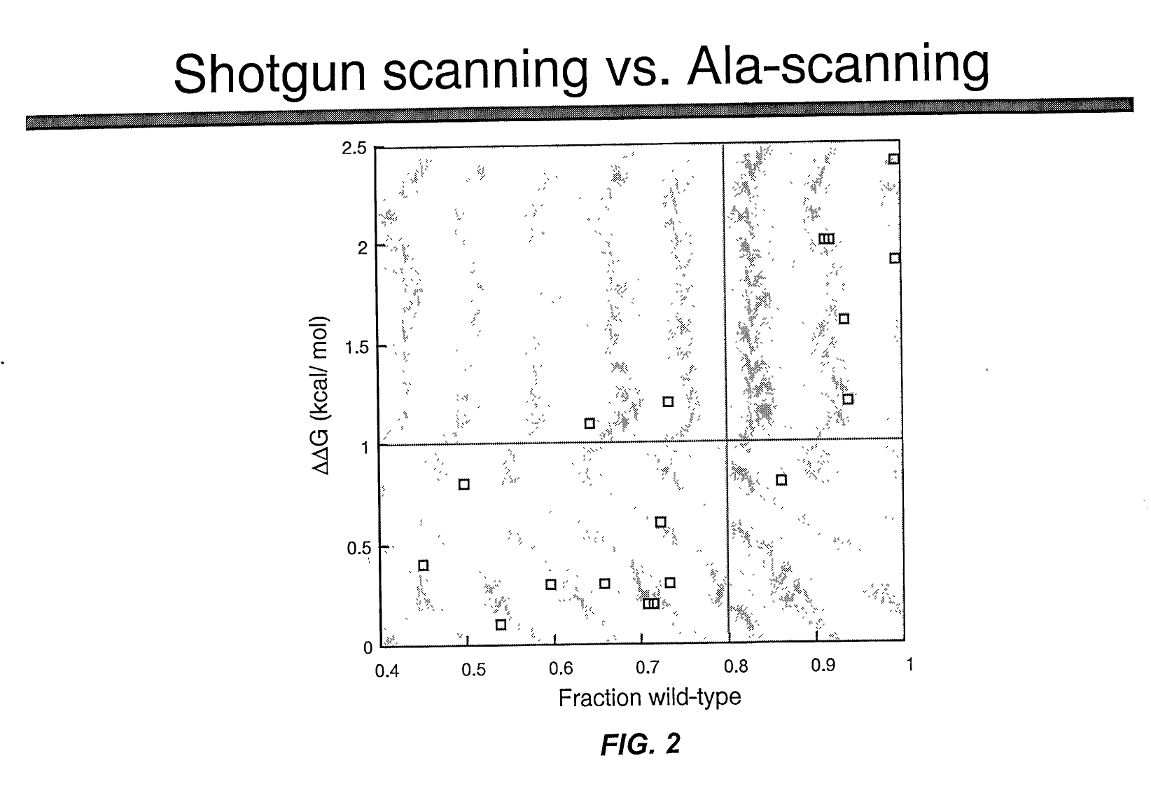

P_{f} values can range from −1 to 1, with negative or positive values indicating unfavorable or favorable contributions to the functional epitope, respectively. Only one side chain (Tyr64) had a negative P_{f} value, and thus the average of all the P_{f} values was positive (P_{f,ave}=0.49, standard deviation=0.35), indicating that most side chains in the hGH structural epitope make favorable contacts with hGHbp. However, the large standard deviation indicated that the side chains in the structural epitope do not contribute equally to the functional binding epitope. Indeed, the P_{f} values formed two distinct clusters, with one cluster containing P_{f} values less than or equal to P_{f,ave} and the second cluster containing P_{f} values significantly greater than P_{f,ave}. The second cluster contains only seven side chains (Pro61, Arg64, Lys172, Thr175, Phe176, Arg178, Ile179), and our results indicate that this subset is mainly responsible for binding affinity. These side chains also cluster together in the three-dimensional structure, and thus form a compact functional binding epitope. Overall, the shotgun scanning results are in good agreement with the results of conventional alanine scanning mutagenesis, which also identified a similar binding epitope (Cunningham and Wells, 1993, J. Mol. Biol. 234:554). The measured P_{f} values were plotted against ΔΔG values (FIG. 2), determined by conventional affinity measurements with individual, purified alanine mutants. Shotgun scanning identified seven of the nine largest binding energy contributors (ΔΔG_{(mut-wt)}≧0.8 kcal/mol).

The few discrepancies between shotgun scanning and alanine-scanning may be due to non-additive interactions between some residues in the shotgun scanning library. In particular, although we ignored all substitutions except alanine and wild-type, it is possible that these additional substitutions skewed the calculated wild-type frequencies at some positions. However, these non-additive effects can be addressed by analyzing co-variation of mutated sites; such analyses can provide information on intramolecular interactions that cannot be obtained from alanine-scanning with single mutants. Also, recent developments in DNA synthesis make it possible to construct libraries in which any site can be restricted to only alanine or one of the other natural amino acids (The single letter abbreviations for amino acid residues are as follows: A, Ala; C, Cys; D, Asp; E, Glu; F, Phe; G, Gly; H, His; I, Ile; K, Lys; L, Leu; M, Met; N, Asn; P, Pro; Q, Gln; R, Arg; S, Ser; T, Thr; V, Val; W, Trp; and Y; Tyr). Shotgun scanning accurately mapped the functional epitope of the hGH site-I binding to hGHbp.

These results demonstrate that shotgun scanning mutagenesis is a robust method well suited for high throughput proteomics. Detailed mapping of protein structure and function is possible without any protein purification or analysis. A high resolution map of a protein binding epitope was obtained from DNA sequence alone, and the results were in excellent agreement with results obtained with conventional protein-based techniques. With the limited diversity of the shotgun code, many positions can be scanned by a single library, and multiple libraries can be used. The method is applicable to proteins, including antibodies, and an entire protein sequence can be rapidly scanned by libraries spanning large stretches of contiguous residues. Identification of binding interaction hot spots expedites protein engineering, through rapid determination of functionally critical residues.

### EXAMPLE 1

### Shotgun Scanning

Experimental: A phagemid pW1205a was constructed using the method of Kunkel (Kunkel et al., 1987, Methods Enzymol. 154:367) and standard well known molecular biology techniques. Phagemid pW1205a was used as the template for library construction. pW1205a is a phagemid for the display of hGH on the surface of filamentous phage particles. In pW1205a, transcription of the hGH-P8 fusion is controlled by the IPTG-inducible Ptac promoter (Amman, E. and Brosius, J., 1985, Gene 40, 183-190). pW1205a is identical to a previously described phagemid designed to display hGH on the surface of M13 bacteriophage as a fusion to the amino terminus of the major coat protein (P8), except for the following changes. The mature P8 encoding DNA segment of pW1205a had the following DNA sequences for codons 11 through 20 (other residues fixed as wild-type):

TAT GAG GCT CTT GAG GAT ATT GCT ACT AAC (SEQ ID NO 1)

This segment encodes the following amino acid sequence:

YEALEDIATN (SEQ ID NO 2).

First, the hGH-P8 fusion moiety has a peptide epitope flag (amino acid sequence: MADPNRFRGKDLGG) (SEQ ID NO 3) fused to its amino terminus, allowing for detection with an anti-flag antibody. Second, codons encoding residues 41, 42, 43, 61, 62, 63, 171, 172, and 173 of hGH have been replaced by TAA stop codons.

Briefly, pW1205a was used as the template for the Kunkel mutagenesis method with three mutagenic oligonucleotides designed to simultaneously repair the stop codons and introduce mutations at the desired sites. The mutagenic oligonucleotides had the following sequences:

| | |
|---|---|
| Oligo1 (mutate hGH codons 41, 42, 45, and 48): | |
| 5′-ATC CCC AAG GAA GAG RMA KMT TCA TTC SYT GAG AAC SCA GAG ACC TCC CTC | (SEQ ID NO 4) |
| TGT TTC-3′ | |
| Oligo2 (mutate hGH codons 61, 62, 63, 64, 67, and 68): | |
| 5′-TCA GAA TCG ATT CGG AGA SCA KCC RMG SST GAG GAA RCT SMA CAG AAA TCC | (SEQ ID NO 5) |
| AAC CTA GAG-3′ | |
| Oligo3 (mutate hGH codons 164, 167, 168, 171, 172, 175, 176, 178, and 179): | |
| 5′-AAG TAC GGG GTG CTC KMY TGC TTC SST RMA GAC ATG GMT RMA GTG GAG RGT | (SEQ ID NO 6) |
| KYT GTG SST RYT GTG CAG TGG GGG TGT-3′ | |

(K=G/T, M=A/C, N=A/C/G/T, R=A/G, S=G/C, W=A/T, Y═C/T). The library contained 1.2×10¹¹ unique members and DNA sequencing of the naive library revealed that 45% of these contained mutations at all the designed positions, thus the library had a diversity of approximately 5.4×10¹⁰.

Procedure 1: In vitro synthesis of heteroduplex DNA. The following three-step procedure is an optimized, large scale version of the method of Kunkel et al. The oligonucleotide was first 5′-phosphorylated and then annealed to a dU-ssDNA phagemid template. Finally, the oligonucleotide was enzymatically extended and ligated to form CCC-DNA.

Step 1: Phosphorylation of the Oligonucleotide

Combine the following in an eppendorf tube:

0.6 μg oligonucleotide

2 μL 10×TM buffer

2 μL 10 mM ATP

1 μL 100 mM DTT

Add water to a total volume of 20 μL. Add 20 units of T4 polynucleotide kinase. Incubate for 1 hour at 37° C.

Step 2: Annealing the Oligonucleotide to the Template

Combine the following in an eppendorf tube:

20 μg dU-ssDNA template

0.6 μg phosphorylated oligonucleotide

25 μL 10×TM buffer

Add water to a total volume of 250 μL. The DNA quantities provide an oligonucleotide:template molar ratio of 3:1, assuming that the oligonucleotide:template length ratio is 1:100.

2. Incubate at 90° C. for 2 min, 50° C. for 3 min, 20° C. for 5 min.

Step 3: Enzymatic Synthesis of CCC-DNA

To the annealed oligonucleotide/template, add the following:

10 μL 10 mM ATP

10 μL 25 mM dNTPs

15 μL 100 mM DTT

30 units T4 DNA ligase (Weiss units)

30 units T7 DNA polymerase

Incubate at 20° C. for at least 3 hours. Affinity purify and desalt the DNA using the Qiagen QIAquick DNA Purification Kit. Follow the manufacturer's instructions. Use one QIAquick column, and elute with 35 μL of ultrapure H₂O.

Electrophorese 1.0 μL of the reaction alongside the single-stranded template. Use a TAE/1.0% agarose gel with ethidium bromide for DNA visualization. A successful reaction results in the complete conversion of single-stranded template to double-stranded DNA. Two product bands are usually visible. The lower band is correctly extended and ligated product (CCC-DNA) which transforms E. coli very efficiently and provides a high mutation frequency (>80%). The upper band is an unwanted product resulting from an intrinsic strand-displacement activity of T7 DNA polymerase. The strand-displaced product provides a low mutation frequency (<20%), but it also transforms E. coli at least 30-fold less efficiently than CCC-DNA. Thus, provided a significant proportion of the template is converted to CCC-DNA, a high mutation frequency will result. Occasionally, a third product band is visible. Migrating between the two bands described above, this band is correctly extended but unligated DNA, resulting either from insufficient T4 DNA ligase activity or from inefficient oligonucleotide phosphorylation. This product must be avoided, because it transforms E. coli efficiently but provides a low mutation frequency.

Procedure 2: Preparation of electrocompetent E. coli SS320. Pick a single colony of E. coli SS320 (from a fresh 2YT/tet plate) into 1 mL of 2YT/tet. Incubate at 37° C. with shaking at 200 rpm for about 8 hours. Transfer the culture to 50 mL of 2YT/tet in a 500-mL baffled flask and grow overnight. Inoculate 5 mL of the overnight culture into six 2-L baffled flasks containing 900 mL of superbroth supplemented with 5 μg/mL tetracycline. Grow cells to an OD₆₀₀ of 0.6-0.8 (approximately 4 hours).

Chill three flasks on ice for 10′ with periodic shaking. All steps from here should be done on ice and in a cold room where applicable. Transfer the cultures to six 400-mL prechilled centrifuge tubes. Centrifuge for 5 min at 5 krpm and 2° C. in a Sorvall GS-3 rotor (5000 g). While the cultures are centrifuging, chill the remaining three flasks on ice. Decant the supernatant and add the cultures from the remaining three flasks to the same centrifuge tubes. Repeat the centrifugation and decant the supernatant.

Fill each tube with 1.0 mM Hepes, pH 7.0. Add a sterile, magnetic stir bar (the stir bars should be rinsed with sterile water before and after use, and they should be stored in ethanol). Use the stir bar to resuspend the pellet: swirl briefly to dislodge the pellet from the tube wall and then stir at a moderate rate until the pellet is completely resuspended. Centrifuge for 10 min at 5 krpm and 2° C. in a GS-3 rotor. When removing the tubes from the rotor, be careful to maintain the angle so as not to disturb the pellet. Decant the supernatant, but do not remove the stir bars. Repeat two previous steps. Resuspend each pellet in 150 mL of 10% glycerol. Do not combine the pellets at this point.

Centrifuge for 15 min at 5 krpm and 2° C. in a GS-3 rotor. Decant the supernatant and remove the stir bars. Remove remaining traces of supernatant with a sterile pipet. Add 3.0 mL of 10% glycerol to the first tube and resuspend the pellet by gently pipetting. Transfer the suspension to another tube and repeat until all the pellets are resuspended. Aliquot 350,L of cells into eppendorf tubes, flash freeze on dry ice, and store at −70° C. The procedure yields approximately 12 mL of cells at a concentration of 3×10¹¹ cfu/m L.

Procedure 3: E. coli electroporation and phage production. Chill the purified DNA and a 0.2-cm gap electroporation cuvet on ice. Thaw a 350 μL aliquot of electrocompetent E. coli SS320 on ice. Add the cells to the DNA and mix by pipetting several times. Transfer the mixture to the cuvet and electroporate. Preferably, use a BTX ECM-600 electroporation system with the following settings: 2.5 kV field strength, 129 ohms resistance, and 50 μF capacitance. Alternatively, a Bio-rad Gene Pulser can be used with the following settings: 2.5 kV field strength, 200 ohms resistance, and 25 μF capacitance.

Immediately add 1 mL of SOC media and transfer to a 250-mL baffled flask. Rinse the cuvet twice with 1 mL SOC media. Add SOC media to a final volume of 25 mL and incubate for 30 min at 37° C. with shaking. Plate serial dilutions on 2YT/carb plates to determine the library diversity. Transfer the culture to a 2-L baffled flask containing 500 mL 2YT/carb/VCS. Incubate overnight at 37° C. with shaking. Centrifuge the culture for 10 min at 10 krpm and 2° C. in a Sorvall GSA rotor (16000 g). Transfer the supernatant to a fresh tube and add 1/5 volume of PEG-NaCl solution to precipitate the phage. Incubate 5 min at room temperature.

Centrifuge for 10 min at 10 krpm and 2° C. in a GSA rotor. Decant the supernatant. Respin briefly and remove the remaining supernatant with a pipet. Resuspend the phage pellet in 1/20 volume of PBS or PBT buffer. Pellet insoluble matter by centrifuging for 5 min at 15 krpm and 2° C. in an SS-34 rotor. Transfer the supernatant to a clean tube. Determine the phage concentration spectrophotometrically (OD₂₆₈=1.0 for a solution containing 5×10¹² phage/mL). Use immediately, or flash freeze on dry ice and store at −70° C.

Procedure 4: Affinity sorting the library. Coat Maxisorp immunoplate wells with 100 μL of target protein solution (2-5 μg/mL in coating buffer) for 2 hours at room temperature or overnight at 4° C. The number of wells required depends on the diversity of the library. Preferably, the phage concentration should not exceed 10¹³ phage/mL and the total number of phage should exceed the library diversity by 1000-fold. Thus, for a diversity of 10¹⁰, 10¹³ phage should be used and, using a concentration of 10¹³ phage/mL, 10 wells will be required.

Remove the coating solution and block for 1 hour with 200 μL of 0.2% BSA in PBS. At the same time, block an equal number of uncoated wells as a negative control. Remove the block solution and wash eight times with PT buffer. Add 100 μL of library phage solution in PBT buffer to each of the coated and uncoated wells. Incubate at room temperature for 2 hours with gentle shaking. Remove the phage solution and wash 10 times with PT buffer. To elute bound phage, add 100 μL of 100 mM HCl. Incubate 5 minutes at room temperature. Transfer the HCl solution to an eppendorf tube. Neutralize with 1.0 M Tris-HCl, pH 8.0 (approximately ⅓ volume). Add half the eluted phage solution to 10 volumes of actively growing E. coli SS320 or XL1-Blue (OD₆₀₀<1.0). Incubate for 20 min at 37° C. with shaking. Plate serial dilutions on 2YT/carb plates to determine the number of phage eluted. Determine the enrichment ratio: the number of phage eluted from a well coated with target protein divided by the number of phage eluted from an uncoated well. Transfer the culture from the coated wells to 25 volumes of 2YT/carb/VCS and incubate overnight at 37° C. with shaking. Isolate phage particles as described in procedure 4. Repeat the sorting cycle until the enrichment ratio has reached a maximum. Typically, enrichment is first observed in round 3 or 4, and sorting beyond round 6 is seldom necessary. Pick individual clones for sequence analysis and phage ELISA.

Solutions and Media

2YT: 10 g bacto-yeast extract, 16 g bacto-tryptone, 5 g NaCl; add water to 1 liter and adjust pH to 7.0 with NaOH; autoclave

2YT/carb: 2YT, 50 μg/mL carbenicillin

2YT/carb/VCS: 2YT/carb, 10¹⁰ pfu/mL of VCSM13

2YT/tet: 2YT, 5 μg/mL tetracycline

10% glycerol: 100 mL of ultrapure glycerol and 900 mL of H₂O; filter sterilized

10×TM buffer: 500 mM Tris-HCl, 100 mM MgCl₂, pH 7.5

coating buffer: 50 mM sodium carbonate, pH 9.6

OPD solution: 10 mg of OPD, 4 μL of 30% H₂O₂, 12 mL of PBS

PBS: 137 mM NaCl, 3 mM KCT, 8 mM Na₂HPO₄, 1.5 mM KH₂PO₄; adjust pH to 7.2 with HCl; autoclave

PEG-NaCl solution: 200 g/L PEG-8000, 146 g/L NaCl; autoclaved

PT buffer: PBS, 0.05% Tween 20

PBT buffer: PBS, 0.2% BSA, 0.1% Tween 20

SOC media: 5 g bacto-yeast extract, 20 g bacto-tryptone, 0.5 g NaCl, 0.2 g KCl; add water to 1.0 liter and adjust pH to 7.0 with NaOH; autoclave; add 5 mL of 2.0 M MgCl₂ (autoclaved) and 20 mL of 1.0 M glucose (filter sterilized).

superbroth: 24 g bacto-yeast extract, 12 g bacto-tryptone, 5 mL glycerol; add water to 900 mL,; autoclave; add 100 mL of 0.17 M KH₂PO₄, 0.72 M K₂HPO₄ (autoclaved).

### EXAMPLE 2

### Serine Shotgun Scan of hGH

A library was constructed using pW1205a as the template, exactly as described in Example 1, except that the following mutagenic oligonucleotides were used:

| | |
|---|---|
| Oligo 1 (mutate hGH codons 41, 42, 45, and 48): | |
| 5′-ATC CCC AAG GAA CAG ARM TMC TCA TTC TYG GAG AAG YCT CAG ACC TCG GTC TGT | (SEQ ID NO 7) |
| TTC-3′ | |
| Oligo 2 (mutate hGH codons 61, 62, 63, 64, 67, 68): | |
| 5′-GAA TCG ATT CCG ACA YCT TCC ARC MGT GAG GAA WGG YMG CAG AAA TCG AAC CTA | (SEQ ID NO 8) |
| GAG-3′ | |
| Oligo 3 (mutate hGH codons 164, 167, 168, 171, 172, 174, 175, 176, 178, 179): | |
| 5′-AAC TAG GGG CTG GTG TMG TGC TTC MGT ARM GAG ATG KMG ARM GTG KMG WCG TYC | (SEQ ID NO 9) |
| GTG MGT AKG GTG GAG TGC CGC TGT-3′ | |

The resulting library contained hGH variants in which the indicated codons were replaced by degenerate codons as described in Table 6. The library contained 2.1×10¹⁰ unique members. The library was sorted against either hGHbp or an anti-hGH antibody as described above and the resulting selectants were analyzed as described above.

For each selection, the ratio of wild-type (wt) to serine at each position was calculated as follows:

wt/Ser=n_{wt}/nₛₑᵣᵢₙₑ

We then determined the ratio of (wt/Ser)_{bp} to (wt/Ser)_{antibody}

This final ratio, (wt/Ser)_{bp}/(wt/Ser)_{antibody} measures the effect on the binding free energy attributable to the mutation of each sidechain to serine. We assumed the following:

(wt/Ser)_{bp}/(wt/Ser)_{antibody}=K_{a,wt}/K_{a,Ser}

Where K_{a,wt} and K_{a,Ser} are the association equilibrium constants for hGHbp binding to wt Or serine-substituted hGH, respectively. With this assumption, we obtained a measure of each serine mutant's effect on the binding free energy by substituting (wt/Ser)_{bp}/(wt/Ser)_{antibody} for K_{a,wt}/K_{a,Ser} in the standard equation:

ΔG_{Ser-wt}=RTln[K_{a,wt}/K_{a,Ser}]=RTln[(wt/Ser)_{bp}/(wt/Ser)_{antibody]}

### EXAMPLE 3

### Homolog Shotgun Scan of hGH

Standard molecular biology techniques were used to construct phagemid pW1269a. Phagemid pW1269a is identical to phagemid pW1205a (example 1) except that codons 14, 15, and 16 of hGH have also been replaced by TAA stop codons.

Phagemid pW1269a was used as the template for the Kunkel mutagenesis method with four oligonucleotides designed to simultaneously repair the stop codons in the hGH gene and introduce mutations at the desired sites. The mutagenic oligonucleotides had the following sequences:

| | |
|---|---|
| Oligo 1 (mutate hGH codons 14, 18, 21, 22, 25, 26, 29): | |
| 5′-ATA CCA CTG TCG AGG CTC KCT GAC AAC GCG TKG CTG CGT GCT GAM CGT GTT RAC SAA CTG GCC | (SEQ ID NO 10) |
| TWG GAM AGG TAC SAA GAG TTT GAA GAA GCC TAT-3′ | |
| Oligo 2 (mutate hGH codons 41, 42, 45, 46, 48): | |
| 5′-ATC CCA AAG GAA CAG RTT MAC TCA TTC TKG TKG AAC YCG CAG ACC TCC CTC TGT CC-3′ | (SEQ ID NO 11) |
| Oligo 3 (mutate hGH codons 61, 62, 63, 64, 65, 68): | |
| 5′-TCA GAG TCT ATT CGG ACA YCG KCC RAC ARG GAM GAA ACA SAA CAG AAA TCC AAC CTA GAG-3′ | (SEQ ID NO 12) |
| Oligo 4 (mutate hGH codons 164, 167, 168, 171, 172, 174, 175, 176, 178, 179, 183): | |
| 5′-AAG AAC TAC GGG TTA CTC TWC TGC TTC RAC ARG GAG ATG KCC ARG GTC KCC ASC TWC CTG ARG | (SEQ ID NO 13) |
| ASC GTG CAG TGC ARG TCT GTG GAG GGC AGC-3′ | |

The resulting library contained hGH variants in which the indicated codons were replaced by degenerate codons as described in Table B. The library contained 1.3×10⁹ unique members. The library was sorted against either hGHbp or an anti-hGH antibody as described above and the resulting selectants were analyzed as described above (see examples 1 and 2). For each mutated position the ΔG_{mut-wt} was determined for each homolog substitution, as described for serine scanning in example 2. The results of this analysis are shown in Table C.

### EXAMPLE 4

### Protein 8 (P8) Shotgun Scan

pS1607 is a previously described phagemid designed to display hGH on the surface of M1:3 bacteriophage as a fusion to the major coat protein (protein-8, P8) (Sidhu S. S., Weiss, G. A. and Wells, J. A. (2000) J. Mol. Biol. 296:487-495). Two phagemids (pR212a and pR212b) were constructed using the Kunkel mutagenesis method with pS1607 as the template. Phagemid pR212a contained TAA stop codons in place of P8 codons 19 and 20, while phagmid pR212b contained TAA stop codons in place of P8 codons 44 and 45.

Three mutagenic oligonucleotides were synthesized as follows:

| | |
|---|---|
| Oligo 1 (mutate P8 residues 1 to 19, inclusive): | |
| 5′-TCC GGG AGC TCC AGC GST GMA GST GMT GMT SCA GST RMA GST GST KYT | (SEQ ID NO 14) |
| RMC KCC SYT SMA GST KCC GST RCT GAA TAT ATC GGT TAT GCG TGG-3′ | |
| Oligo 2 (mutate P8 residues 20 to 36, inclusive): | |
| 5′-CTG CAA GCC TCA GCG ACC GMA KMT RYT GST KMT GST KSG GST RYG GYT | (SEQ ID NO 15) |
| GYT GYT RYT GYT GST GST RCT ATC GGT ATC AAG CTG TTT-3′ | |
| Oligo 3 (mutate P8 residues 37 to 50, inclusive): | |
| 5′-ATT GTC GGC GCA ACT RYT GST RYT RMA SYT KYT RMA RMA KYT RCT KCC | (SEQ ID NO 16) |
| RMA GST KCC TGA TAA ACC GAT ACA ATT-3′ | |

pR212a was used as the template for the Kunkel mutagenesis method with Oligo 1 to produce a library with mutations introduced at P8 positions 1 to 19, inclusive. Similarly, Oligo 2 was used to construct a library with mutations at P8 positions 20 to 36, inclusive. Finally, pR212b was used as the template with Oligo 3 to construct a third library with mutations introduced at P8 positions 37 to 50, inclusive. In each library, the mutated codons were replaced by degenerate codons as shown in Table 1.

Each library was sorted to select members that bound to hGHbp, as described above. Positive clones were identified, sequenced, and analyzed as described above. For each position in P8, the ratio of wt/mutant was determined, where mutant is either glycine (when wt is alanine) or alanine (for all other wt amino acids). The results of this analysis are shown in Table D.

The wt/mutant ratio indicates the importance of a particular sidechain for incorporation of P8 into the phage coat. If wt/mutant is greater than 1.0, the wt sidechain contributes favorably to incorporation. Conversely, if wt/mutant is less than 1.0, the wt sidechain contributes unfavorably to incorporation.

### EXAMPLE 5

### Anti-Her2 Fab-2C4 Alanine Shotgun Scan

A phagemid vector (designated S74.C11) was constructed to display Fab-2C4 on M13 bacteriophage with the heavy chain fused to the N-terminus of the C-terminal domain of the gene-3 minor coat protein (P3) (see Cam Adams). The light chain was expressed free in solution and functional Fab display resulted by the assembly of free light chain with phage-displayed heavy chain. Also, the light chain had an epitope tag (MADPNRFRGKDL) (SEQ ID NO 17) fused to its N-terminus to permit detection and selection with an anti-tag antibody (anti-tag antibody-3C8).

Part A: Light Chain Scan

Standard molecular biology techniques were used to replace Fab-2C4 light chain codons 27, 28, 50, 51, 91, and 92 with TAA stop codons; the new phagemid was named pS-1655a.

The following mutagenic oligonucleotides were synthesized:

| | |
|---|---|
| Oligo 1 (mutate Fab-2G4 codons 27, 28, 30, 31, and 32 in light chain GDR-1): | |
| 5′-ACG TGC AAG GCG AGT SMA GMT GTG KCC RYT GST GTC GCC TGG TAT GAA-3′ | (SEQ ID NO 18) |
| Oligo 2 (mutate Fab-2G4 codons 50, 52, 53, and 55 in light chain CDR-2): | |
| 5′-AAA GTA CTG ATr TAC KGC GCT KCC KMT CGA KMT ACT GGA GTC CGT TCT-3′ | (SEQ ID NO 19) |
| Oligo 3 (mutate Fab-2C4 codons 91, 92, 93, 94, and 96 in light chain CDR-3): | |
| 5′-TAT TAC TGT CAA GAA KMT KMT RYT KMT GGT KMT ACG YET GGA GAG GGT-3′ | (SEQ ID NO 20) |
| Oligo 4 (mutate Fab-2C4 codons 24, 26, 29, and 33 in light chain GDR-1): | |
| 5′-GTC ACC ATC ACC TGC RMA GST KCC CAG GAT GYT TGT ATT GGT GYT GST TGG TAT | (SEQ ID NO 21) |
| CAA CAG AAA CCA-3′ | |
| Oligo 5 (mutate Fab-2G4 codons 51, 54 and 56 in light chain GDR-2): | |
| 5′-AAA CTA CTG ATT TAC TCG GST TCC TAC SST TAC RGT GGA GTC CCT TCT CGC-3′ | (SEQ ID NO 22) |
| Oligo 6 (mutate Fab-2G4 codons 89, 90, 95, and 97 in light chain GDR-3): | |
| 5′-GCA ACT TAT TAC TGT SMA SMA TAT TAT ATT TAT SCA TAC RCT TTT GGA CAG GGT | (SEQ ID NO 23) |
| ACC-3′ | |

The Kunkel mutagenesis method was used to construct two libraries, using pS1655a as the template. For library 1, Oligos 1, 2, and 3 were used simultaneously to repair the TAA stop codons in pS1655a and replace the indicated codons with degenerate codons as shown in Table 1. Library 1 contained 1.4×10¹⁰ unique members. Library 2 was constructed similarly except that Oligos 4, 5, and 6 were used; library 2 contained 2.5×10¹⁰ unique members.

Each library was sorted separately against either Her2 or anti-tag antibody-3C8. The resulting selectants were analyzed as described in example 2, above. For each position, the ratio (wt/Ala)_{Her2}/(wt/Ala)_{antibody} was determined and used to assess the importance of each sidechain to the binding interaction with Her2 antigen. A ratio greater than one indicates positive contributions to binding while a ratio less than one indicates negative contributions to binding. In this case, the anti-tag antibody-3C8 sort was used to correct for effects on Fab display levels due to mutations, since this antibody detects displayed Fab levels but does not bind to the Fab itself (instead, it binds to the epitope tag fused to the light chain). The results of this analysis are shown in Table E.

Part B: Heavy Chain Scan

Standard molecular biology techniques were used to replace Fab-2C4 heavy chain codons 28, 29, 50, 51, 99, and 100 with TAA stop codons; the new phagemid was named pS-1655b.

The following mutagenic oligonucleotides were synthesized:

| | |
|---|---|
| Oligo 1 (mutate Fab-2C4 codons 28, 30, 31, 32, and 33 in heavy chain CDR-1): | |
| 5′-GCA GCT TCT GGC TTG RCT TTC RCT GMT KMT RCT ATG GAC TGG GTC CGT-3′ | (SEQ ID NO 24) |
| Oligo 2 (mutate Fab-2C4 codons 50, 51, 52, 54, 55, 59, 61, and 62 in heavy chain CDR-2): | |
| 5′-CTG GAA TGG GTT GCA GMT GYT RMC CCT RMC KCC GGC GGC TGT RYT TAT RMC SMA CGC TTC AAG | (SEQ ID NO 25) |
| GGC CGT-3′ | |
| Oligo 3 (mutate Fab-2C4 codons 99, 100, 102, and 103 in heavy chain CDR-3): | |
| 5′-TAT TAT TGT GCT CGT RMC SYT GGA SCA KCC TTC TAC TTT GAC TAC-3′ | (SEQ ID NO 26) |
| Oligo 4 (mutate Fab-2C4 codon 35 in heavy chain CDR-1): | |
| 5′-GCA GCT TCT GGC TTC ACC TTC ACC GAC TAT ACC ATG GMT TGG GTC CGT CAG GCC-3′ | (SEQ ID NO 27) |
| Oligo 5 (mutate Fab-2C4 codons 53, 56, 57, 58, 60, 63, 64, 65, and 66 in | |
| heavy chain CDR-2): | |
| 5′-CTG GAA TGG GTT GCA GAT GTT AAT SCA AAC AGT GST GST KCC ATC KMT AAC CAG SST KYT RMA | (SEQ ID NO 28) |
| GST CGT TTC ACT CTG AGT-3′ | |
| Oligo 6 (mutate Fab-2G4 codons 101, 104, 105, 106, 107, and 108 in heavy chain GDR-3): | |
| 5′-TAT TAT TGT GCT CGT AAC CTG GST CCC TCT KYT KMT KYT GMT KMT TGG GGT CAA GGA ACC-3′ | (SEQ ID NO 29) |

Two libraries were constructed, sorted and analyzed as described in Part A, above. For the construction of library 1, phagemid pS 1655b was used as the template for the Kunkel mutagenesis method with Oligos 1, 2, and 3. Similarly, library 2 was constructed with Oligos 4, 5, and 6. Library 1 contained 4.6×10¹⁰ unique members and library 2 contained 2.4×10¹⁰ unique 40 members. The results of the analysis are shown in Table F.

### EXAMPLE 6

### Anti-Her2 Fab-2C4 Homolog Scan

This scan was conducted as described in example 5, except the scanned residues were mutated according to the “homolog shotgun code” shown in Table B.

Part A: Light Chain Scan

The following mutagenic oligonucleotides were synthesized:

| | |
|---|---|
| Oligo 1 (mutate Fab-2C4 codons 24 to 34 in light chain CDR-1): | |
| 5′-GTC ACC ATC ACC TGC ARG KCC KCC SAA GAM RTT KCC RTT GST | (SEQ ID NO 30) |
| RTT KCC TGG TAT CAA CAG AAA CCA-3′ | |
| Oligo 2 (mutate Fab-2C4 codons 50 to 56 in light chain CDR-2): | |
| 5′-AAA CTA GTG ATT TAC KCC KCC KCC TWC ARG TWC ASC GGA GTC | (SEQ ID NO 31) |
| CCT TCT CCG-3′ | |
| Oligo 3 (mutate Fab-2G4 codons 89 to 97 in light chain GDR-3): | (SEQ ID NO 32) |
| 5′-GCA ACT TAT TAC TGT SAA SAA TWC TWC RTT TWC SCA TWC ASC | |
| TTT GGA CAG GGT AGC-3′ | |

A library was constructed using the Kunkel mutagenesis method with pS1655a as the template and Oligos 1, 2, and 3. The library contained 2.4×10¹⁰ unique members. The library was sorted and analyzed as described in example 5, above. The results of the analysis are shown in Table G.

Part B: Heavy Chain Scan

The following oligonucleotides were synthesized:

| | |
|---|---|
| Oligo 1 (mutate Fab-2C4 codons 28 and 30 to 35 in heavy chain GDR-1): | |
| 5′-GCA GCT TCT GGC TTC ASC TTC ASC GAM TWC ASC MTG GAM TGG GTC CGT CAG GCC-3′ | (SEQ ID NO 33) |
| Oligo 2 (mutate Fab-2C4 codons 50 to 66 in heavy chain GDR-2): | |
| 5′-GGC CTG GAA TGG GTT GCA GAM RTT RAC SCA RAC KGG GST GST KCC RTT TWC RAC | (SEQ ID NO 34) |
| SAA ARG TWC ARG GST GGT TTC ACT CTG AGT-3′ | |
| Oligo 3 (mutate Fab-2C4 codons 99 to 108 in heavy chain CDR-3): | |
| 5′-TAT TAT TGT GCT CGT RAC MTC GST SCA KCC TWC TWC TWC GAM TWG TGG GGT GAA | (SEQ ID NO 35) |
| GGA ACC-3′ | |
| Oligo 4 (produce wild-type sequence in Fab-2C4 heavy chain CDR-1): | |
| 5′-GCA GCT TCT GGC TTC ACC TTT AAC GAC TAT ACC ATG-3′ | (SEQ ID NO 36) |
| Oligo 5 (produce wild-type sequence in Fab-2C4 heavy chain CDR-2): | |
| 5′-CTG GAA TGG GTT GCA GAG GTT AAT CCT AAC AGT GGC-3′ | (SEQ ID NO 37) |
| Oligo 6 (produce wild-type sequence in Fab-2C4 heavy chain GDR-3): | |
| 5′-TAT TAT TGT GCT CGT AAC CTG GGA CCC TCT TTC TAC-3′ | (SEQ ID NO 38) |

Two libraries were constructed using the Kunkel mutagenesis method with pS 1655b as the template. Library 1 used Oligos 2, 4, and 6 which repaired heavy chain CDR-1 and CDR-3 to the wild-type Fab-2C4 sequence and mutated heavy chain CDR-2, as described above. Library 1 contained 2.2×10¹⁰ unique members. Library 2 used Oligos 1, 3, and 5 which repaired heavy chain CDR-2 to the wild-type Fab-2C4 sequence and mutated heavy chain CDR-1 and CDR-3, as described above. Library 2 contained 2.4×10¹⁰ unique members. The libraries were sorted and analyzed as described in example 5, above. The results of the analysis are shown in Table H.

TABLE A
hGH Serine Scan

| | | | (wt/ser)_{bp} | ΔΔG_{Ser-wt} |
|---|---|---|---|---|
| wt aa | (wt/Ser)_{bp} | (wt/Ser)_{antibody} | (wt/Ser)_{antibody} | (kcal/mol) |
| K41 | 1.31 | 0.71 | 0.60 | −0.30 |
| Y42 | 1.14 | 0.66 | 1.73 | 0.33 |
| L45 | 3.70 | 2.21 | 1.67 | 0.30 |
| P48 | 1.91 | 1.25 | 1.53 | 0.25 |
| P61 | 3.52 | 0.63 | 5.59 | 1.02 |
| N63 | 0.43 | 0.71 | 0.61 | −0.29 |
| R64 | 5.14 | 1.67 | 3.08 | 0.67 |
| T67 | 5.58 | 2.07 | 2.70 | 0.59 |
| Q68 | 2.02 | 1.11 | 1.82 | 0.36 |
| Y164 | 1.30 | 1.39 | 0.94 | −0.04 |
| R167 | 1.25 | 0.75 | 1.67 | 0.30 |
| K168 | 0.87 | 1.19 | 0.73 | −0.19 |
| D171 | 0.40 | 0.67 | 0.60 | −0.30 |
| K172 | 3.12 | 0.46 | 6.78 | 1.14 |
| E174 | 0.97 | 0.89 | 1.10 | 0.06 |
| T175 | 1.20 | 0.45 | 2.67 | 0.58 |
| F176 | 22.19 | 4.06 | 5.47 | 1.01 |
| R178 | 6.53 | 1.02 | 6.40 | 1.10 |
| I179 | 2.65 | 0.61 | 4.34 | 0.87 |

TABLE B
Homolog shotgun code

| Amino acid | Shotgun codon | Substitutions |
|---|---|---|
| A | KCT | A/S |
| C | TSC | C/S |
| D | GAM | D/E |
| E | GAM | E/D |
| F | TWC | F/Y |
| G | GST | G/A |
| H | MAC | H/N |
| I | RTT | I/V |
| K | ARG | K/R |
| L | MTC | L/U |
| M | MTG | M/L |
| N | RAC | N/D |
| P | SCA | P/A |
| Q | SAA | Q/E |
| R | ARG | R/K |
| S | KCC | S/A |
| T | ASC | T/S |
| V | RTT | V/I |
| W | TKG | W/L |
| Y | TWC | Y/F |

TABLE C
hGH homolog scan

| | | | (wt/mut)_{bp} | ΔΔG_{mut-wt} |
|---|---|---|---|---|
| mutation | (wt/mut)_{bP} | (wt/mut)_{antibody} | (wt/mut)_{antibody} | (kcal/mol) |
| M14L | 1.47 | 1.83 | 0.80 | −0.13 |
| H18N | 1.18 | 1.26 | 0.94 | −0.04 |
| H21N | 1.64 | 0.74 | 2.22 | 0.47 |
| Q22E | 1.07 | 0.86 | 1.24 | 0.13 |
| F25Y | 1.14 | 0.86 | 1.33 | 0.17 |
| D26E | 1.86 | 1.65 | 1.13 | 0.07 |
| Q29E | 1.62 | 1.04 | 1.56 | 0.26 |
| K41R | 4.26 | 0.86 | 4.95 | 0.95 |
| Y42F | 1.19 | 0.86 | 1.38 | 0.19 |
| L45I | 1.87 | 1.83 | 1.02 | 0.01 |
| Q46E | 4.26 | 1.16 | 3.67 | 0.77 |
| P48A | 0.56 | 0.56 | 1.00 | 0.00 |
| P61A | 10.63 | 0.43 | 24.72 | 1.90 |
| S62A | 1.19 | 1.04 | 1.14 | 0.08 |
| N63D | 2.96 | 0.73 | 4.05 | 0.83 |
| R64K | 0.63 | 1.16 | 0.54 | −0.37 |
| E65D | 0.73 | 0.74 | 0.99 | 0.00 |
| Q68E | 2.34 | 1.16 | 2.02 | 0.42 |
| Y164F | 1.75 | 1.30 | 1.35 | 0.18 |
| R167K | 1.08 | 1.45 | 0.74 | −0.18 |
| K168R | 0.49 | 0.50 | 0.98 | −0.01 |
| D171E | 14.25 | 1.12 | 12.72 | 1.51 |
| K172R | 1.36 | 0.96 | 1.42 | 0.21 |
| E174D | 0.81 | 0.61 | 1.33 | 0.17 |
| T175S | 3.74 | 0.50 | 7.48 | 1.19 |
| F176Y | 1.36 | 1.08 | 1.26 | 0.14 |
| R178K | 5.00 | 2.12 | 2.36 | 0.51 |
| I179V | 0.29 | 0.50 | 0.58 | −0.32 |
| R183K | 4.87 | 0.79 | 6.16 | 1.08 |
| | | | | 10.19 |

TABLE D
P8 shotgun scan

| | wt/mutant |
|---|---|
| 1A | 0.91 |
| 2E | 0.76 |
| 3G | 1.9 |
| 4D | 1.3 |
| 5D | 2.5 |
| 6P | .85 |
| 7A | 7.1 |
| 8K | 1.1 |
| 9A | 6.0 |
| 10A | 56 |
| 11F | >168 |
| 12N | 0.82 |
| 13S | 0.28 |
| 14L | 150 |
| 15Q | .40 |
| 16A | 1.7 |
| 17S | 0.25 |
| 18A | 6.1 |
| 19T | 0.64 |
| 20E | 2.9 |
| 21Y | 1.5 |
| 22I | 0.46 |
| 23G | 3.4 |
| 24Y | 7.0 |
| 25A | 18 |
| 26W | 1.5 |
| 27A | 0.55 |
| 28M | 1.1 |
| 29V | 0.26 |
| 30V | 1.9 |
| 31V | 0.71 |
| 32I | 0.27 |
| 33V | 0.48 |
| 34G | 1.6 |
| 35A | 4.6 |
| 36T | 1.2 |
| 37I | 1.0 |
| 38G | 0.83 |
| 39I | 103 |
| 40K | 54 |
| 41L | 6.8 |
| 42F | 13 |
| 43K | 81 |
| 44K | 20 |
| 45F | 80 |
| 46T | 1.4 |
| 47S | 4.6 |
| 48K | 0.84 |
| 49A | 3.S |
| 50S | 5.0 |

TABLE E
Fab-2C4 Light chain alanine shotgun scan

| | | | (wt/Ala)_{Her2} |
|---|---|---|---|
| position | (wt/Ala)_{Her2} | (wt/Ala)_{antibody} | (wt/Ala)_{antibody} |
| K24 | 0.89 | 0.42 | 2.1 |
| S26 | 3.53 | 2.94 | 1.2 |
| Q27 | .67 | .88 | 0.76 |
| D28 | 1.11 | 0.99 | 1.12 |
| V29 | 6.08 | 2.52 | 2.4 |
| S30 | 1.75 | 1.54 | 1.14 |
| I31 | .91 | 1.71 | 0.53 |
| G32 | 3.30 | 2.89 | 1.14 |
| V33 | 15.80 | 3.29 | 4.8 |
| S50 | 1.02 | 1.32 | 0.77 |
| S52 | 1.30 | 1.53 | 0.85 |
| Y53 | 1.9 | 1.56 | 1.22 |
| R54 | 3.15 | 1.73 | 1.8 |
| Y55 | 31.8 | 1.38 | 23.1 |
| T56 | 0.49 | 0.89 | 0.6 |
| Q89 | 8.75 | 0.77 | 11.4 |
| Q90 | 2.40 | 0.88 | 2.7 |
| Y91 | >166 | 1.8 | >92 |
| Y92 | 1.22 | 1.27 | 0.96 |
| I93 | 1.71 | 1.68 | 1.02 |
| Y94 | 6.72 | 1.87 | 3.6 |
| P95 | 13.17 | 1.09 | 12.0 |
| Y96 | 0.99 | 2.07 | 0.48 |
| T97 | 0.56 | 0.89 | 0.6 |

TABLE F
Fab-2C4 Heavy chain alanine shotgun scan

| | | | (wt/Ala)_{Her2} |
|---|---|---|---|
| position | (wt/Ala)_{Her2} | (wt/Ala)_{antibody} | (wt/Ala)antibody |
| T28 | 4.48 | 0.7 | 6.4 |
| T30 | 0.33 | 0.7 | 0.47 |
| D31 | 170 | 1.4 | 121 |
| Y32 | >161 | 2.0 | >81 |
| T33 | 20.1 | 0.94 | 21.4 |
| D35 | 2.8 | 0.14 | 20 |
| D50 | 170 | 0.24 | 708 |
| V51 | 10.3 | 1.1 | 9.4 |
| N52 | >168 | 0.41 | >410 |
| P53 | 72 | 6.1 | 12 |
| N54 | >166 | 1.4 | >119 |
| S55 | 84 | 0.33 | 255 |
| G56 | 13.6 | 0.4 | 34 |
| G57 | 0.6 | 0.2 | 3 |
| S58 | 7 | 4.4 | 1.6 |
| I59 | 45.3 | 0.86 | 53 |
| Y60 | 33 | 8.7 | 3.8 |
| N61 | 4.8 | 1.2 | 4.0 |
| G62 | 2.55 | 0.53 | 4.8 |
| R63 | 4.3 | 1.2 | 3.6 |
| F64 | 29 | 6.6 | 4.4 |
| K65 | 61 | 4.9 | 12 |
| G66 | 5.8 | 0.4 | 15 |
| N99 | >176 | 1.8 | >98 |
| L100 | 22.5 | 0.11 | 205 |
| G101 | >78 | 3.3 | >24 |
| P102 | >178 | 1.9 | >94 |
| S103 | 2.76 | 0.55 | 5.0 |
| F104 | >75 | 2.4 | >31 |
| Y105 | >74 | 0.8 | >93 |
| F106 | 77 | 2.6 | 30 |
| D107 | 9.1 | 1.1 | 8.3 |
| Y108 | 8.3 | 2.3 | 3.6 |

TABLE G
Fab-2C4 Light chain homolog scan

| | | | (wt/mut)_{Her2} |
|---|---|---|---|
| mutation | (wt/mut)_{Her2} | (wt/mut)_{antibody} | (wt/mut)_{antibody} |
| K24R | 0.88 | 1.02 | 0.9 |
| A25S | 2.76 | 1.56 | 1.8 |
| S26A | 2.82 | 1.48 | 1.9 |
| Q27E | 0.51 | 0.73 | 0.7 |
| D28E | 1.84 | 1.85 | 1.0 |
| V29I | 3.50 | 1.96 | 1.8 |
| S30A | 1.10 | 0.87 | 1.3 |
| I31V | 0.64 | 0.55 | 1.2 |
| G32A | 4.82 | 3.88 | 1.2 |
| V33I | 3.06 | 2.77 | 1.1 |
| A34S | 5.50 | 2.50 | 2.2 |
| S50A | 0.78 | 0.87 | 0.9 |
| A51S | 1.56 | 0.85 | 1.8 |
| S52A | 1.21 | 1.72 | 0.7 |
| Y53F | 1.37 | 1.26 | 1.1 |
| R54K | 3.00 | 2.35 | 1.3 |
| Y55F | 4.82 | 0.95 | 5.1 |
| T56S | 0.88 | 0.76 | 1.2 |
| Q89E | 3.57 | 1.93 | 1.8 |
| Q90E | 0.67 | 0.71 | 0.9 |
| Y91F | 0.94 | 1.24 | 0.8 |
| Y92F | 0.88 | 0.60 | 1.5 |
| I93V | 0.69 | 0.53 | 1.3 |
| Y94F | 1.29 | 0.63 | 2.0 |
| P95A | 9.67 | 1.74 | 5.6 |
| Y96F | 0.36 | 0.91 | 0.4 |
| T97S | 0.28 | 0.35 | 0.8 |

TABLE H
Fab-2C4 Heavy chain homolog shotgun scan

| | | | (wt/mut)_{Her2} |
|---|---|---|---|
| mutation | (wt/mut)_{Her2} | (wt/mut)_{antibody} | (wt/mut)_{antibody} |
| T28S | 0.94 | 0.47 | 2.0 |
| T30S | 0.27 | 0.39 | 0.7 |
| D31E | 29 | 1.1 | 26 |
| Y32F | 17 | 0.85 | 20 |
| T33S | 8.9 | 0.38 | 23 |
| M34L | 2.2 | 0.88 | 2.5 |
| D35E | 14 | 0.90 | 15 |
| D50E | >91 | 0.41 | >222 |
| V51I | 1.28 | 1.75 | 0.73 |
| N52D | >91 | 0.83 | >110 |
| P53A | 14.2 | 0.62 | 22.9 |
| N54D | >91 | 0.57 | >160 |
| S55A | >91 | 1.10 | >83 |
| G56A | 90 | 2.91 | 30.9 |
| G57A | 0.36 | 2.55 | 0.14 |
| S58A | 0.47 | 0.86 | 0.55 |
| I59V | 1.60 | 0.86 | 1.86 |
| Y60F | 0.78 | 0.58 | 1.34 |
| N61D | 2.96 | 1.79 | 1.65 |
| G62A | 0.69 | 0.71 | 0.97 |
| R63K | 1.25 | 1.22 | 1.02 |
| F64F | 3.24 | 4.00 | 0.81 |
| K65R | 0.57 | 0.67 | 0.85 |
| G66A | 9.11 | 3.88 | 2.35 |
| N99 | 21.3 | 3.1 | 6.9 |
| L100 | 1.5 | 1.2 | 1.3 |
| G101 | 89 | 2.1 | 42 |
| P102 | 28.7 | 0.44 | 65 |
| S103 | 7.0 | 1.6 | 4.4 |
| F104 | 10 | 1.1 | 9.1 |
| Y105 | 1.7 | 0.49 | 3.5 |
| F106 | 16.6 | 5.1 | 3.3 |
| D107 | >87 | 2.5 | >35 |
| Y108 | 2.8 | 0.92 | 3.0 |

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

| pos | wild | | | | | | | | | | |
|---|---|---|---|---|---|---|---|---|---|---|---|
| X | total | A | C | D | E | F | . . . | V | W | Y | O |
| 30 | E | 0 | 0 | 0 | 89 | 0 | . . . | 0 | 0 | 0 | 0 |
| 0 | 89 | | | | | | | | | | |
| 31 | F | 0 | 0 | 0 | 0 | 89 | . . . | 1 | 0 | 0 | 0 |
| 0 | 90 | | | | | | | | | | |
| . . . | | | | | | | | | | | |

While the invention has necessarily been described in conjunction with preferred embodiments, one of ordinary skill, after reading the foregoing specification, will be able to effect various changes, substitutions of equivalents, and alterations to the subject matter set forth herein, without departing from the spirit and scope thereof. Hence, the invention can be practiced in ways other than those specifically described herein. It is therefore intended that the protection granted by Letters Patent hereon be limited only by the appended claims and equivalents thereof.

All patent and literature references cited above are incorporated herein by reference in their entirety.

What is claimed:

1. A library comprising fusion genes encoding a plurality of fusion proteins, wherein the fusion proteins comprise a polypeptide portion fused to at least a portion of a phage coat protein, the polypeptide portion of the fusion proteins differ at a predetermined number of amino acid positions, and the fusion genes encode at most eight different amino acids at each predetermined amino acid position.

2. A library comprising expression vectors containing fusion genes encoding a plurality of fusion proteins, wherein the fusion proteins comprise a polypeptide portion fused to at least a portion of a phage coat protein, the polypeptide portion of the fusion proteins differ at a predetermined number of amino acid positions, and the fusion genes encode at most eight different amino acids at each predetermined amino acid position.

3. A library comprising phage or phagemid particles displaying a fusion protein on the surface thereof and containing fusion genes encoding a plurality of fusion proteins, wherein the fusion proteins comprise a polypeptide portion fused to at least a portion of a phage coat protein, the polypeptide portion of the fusion proteins differs at a predetermined number of amino acid positions, and the fusion genes encode at most eight different amino acids at each predetermined amino acid position.

4. The library of any one of claims 1-3, wherein the fusion genes encode only a wild type amino acid, a single scanning amino acid and optionally two non-wild type, non-scanning amino acids at each predetermined amino acid position.

5. The library of any one of claims 1-3, wherein the fusion genes encode only a wild type amino acid and a single scanning amino acid at one or more predetermined amino acid position.

6. The library of any one of claims 1-3, wherein the fusion genes encode only a wild type amino acid and a single scanning amino acid at each predetermined amino acid position.

7. The library of any one of claims 1-3, wherein the fusion genes encode only a wild type amino acid and a homolog scanning amino acid at one or more predetermined amino acid position.

8. The library of any one of claims 1-3, wherein the fusion genes encode only a wild type amino acid and a homolog scanning amino acid at each predetermined amino acid position.

9. The library of any of the preceding claims, wherein the fusion genes encode a scanning amino acid selected from the group consisting of alanine, cysteine, phenylalanine, proline, isoleucine, serine, glutamic acid and arginine at the predetermined amino acid position.

10. The library of any of the preceding claims, wherein the fusion genes encode at least alanine at the predetermined amino acid position.

11. The library of any of the preceding claims, wherein the phage coat protein is a filamentous phage coat protein.

12. The library of any of the preceding claims, wherein the phage coat protein is M13 phage coat protein 3 or 8.

13. The library of any of the preceding claims, wherein the predetermined number is in the range 2-60, preferably 5-40, more preferably, 5-35.

14. Host cells comprising the library of any of the preceding claims.

15. A method, comprising the steps of:
constructing the library of particles of any one of claims 3-13;
contacting the library of particles with a target molecule so that at least a portion of the particles bind to the target molecule; and
separating the particles that bind from those that do not bind.

16. The method of claim 15, further comprising determining the ratio of wild-type:scanning amino acids at one or more, preferably all, of the predetermined positions for at least a portion of polypeptides on the particles which bind or which do not bind.

17. The method of claim 15 or 16, wherein the polypeptide and target molecule are selected from the group of polypeptide/target molecule pairs comprising ligand/receptor, receptor/ligand. ligand/antibody and antibody/ligand.

18. A method for producing a product polypeptide, comprising the steps of:
(1) culturing a host cell transformed with a replicable expression vector, the replicable expression vector comprising DNA encoding a product polypeptide operably linked to a control sequence capable of effecting expression of the product polypeptide in the host cell; wherein the DNA encoding the product polypeptide has been obtained by a method comprising the steps of:
(a) constructing a library of expression vectors of any of claims 2, 4-13;
(b) transforming suitable host cells with the library of expression vectors;
(c) culturing the transformed host cells under conditions suitable for forming recombinant phage or phagemid particles displaying variant fusion proteins on the surface thereof;
(d) contacting the recombinant particles with a target molecule so that at least a portion of the particles bind to the target molecule;
(e) separating particles that bind to the target molecule from those that do not bind;
(f) selecting one of the variant as the product polypeptide and cloning DNA encoding the product polypeptide into the replicable expression vector; and
(2) recovering the expressed product polypeptide.

19. The method of claim 18, wherein (f) further comprises mutating the selected variant to form a mutated variant and selecting the mutated variant as the product polypeptide.

20. A method of determining the contribution of individual amino acid side chains to binding of a polypeptide to a ligand therefor, comprising
constructing a library of particles of any one of claims 3-13;
contacting the library of particles with a target molecule so that at least a portion of the particles bind to the target molecule; and
separating the particles that bind from those that do not bind.

21. The method of claim 20, wherein a wild type amino acid and a scanning amino acid are encoded at each predetermined amino acid position and further comprising determining the ratio of wild-type:scanning amino acid at one or more, preferably all, of the predetermined positions for at least a portion of polypeptides on the particles which bind or which do not bind.
